(19) United States
(12) Patent Application Publication
(10) Pub. No.: US 2007/0231412 A1
(43) Pub. Date: Oct. 4, 2007

(54) Virus-interacting layered phyllosilicates and methods of inactivating viruses in the gastrointestinal tract

(75) Inventors: John Hughes, University Park, FL (US); Panayiotis P. Constantinides, Gurnee, IL (US); Jerald W. Darlington JR., Marengo, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMCOL INTERNATIONAL, Arlington Heights, IL (US)

(21) Appl. No.: 11/641,639

(22) Filed: Dec. 18, 2006

Related U.S. Application Data
(63) Continuation-in-part of application No. 11/196,090, filed on Aug. 3, 2005.

(51) Int. Cl. A61K 48/00; A61K 33/06; A61K 33/12; A61K 33/38

(52) U.S. Cl. 424/683; 424/684; 514/44; 424/618

(57) ABSTRACT

Layered phyllosilicates are useful for adsorbing and/or binding to and, thereby, inactivating viruses. Accordingly, provided herein is a method of inactivating a virus in the gastrointestinal tract of a mammalian subject comprising administering to said subject a composition comprising a layered phyllosilicate material in an amount effective for virus inactivation. Also provided are methods of treating a viral infection in the gastrointestinal tract of a mammalian subject. Methods of delivering a therapeutic agent to a mammalian subject and methods of inactivating a virus in waste expelled from a mammal are also provided.

This application is a continuation-in-part of U.S. patent application Ser. No. 11/196,090, filed Aug. 3, 2005, the disclosure of which is incorporated herein by reference in its entirety.

### FIELD

Described herein are virucidal layered phyllosilicates capable of interacting with and thereby inactivating significant percentages of bacteria and a plurality of viruses.

### BACKGROUND

The number of people who were infected with HIV rose to its highest level ever in 2004. The WHO estimated a global total of 39.4 million people living with HIV and that 3.1 million people died of the infection in 2004 (www.unaids.org/wad2004/report.html). Of the world's HIV-infected individuals 50% with teenage girls accounting for 30% of the HIV infected women in some sub-Saharan African countries. Although contraception is available, the HIV epidemic continues to spread highlighting the urgent need for new prevention strategies (Balzarini, J. 2005). Virucides are of interest because they can act quickly and are more direct by binding to the virus coat proteins or viral membranes on contact (Al-Jabri, A. A et al., 2000). A number of HIV virucides are currently under investigation including the physical method of absorbing the virus using mineral clays, a method tried and tested by a number of scientists (Quignon, F. et al. 1997; Clark, K. J., Sarr, A. B., Grant, P. G., Phillips, T. D. & Woode, G. N., 1998; Meschke, J. S. & Sobsey, M. D., 2003). The adsorption effects of bentonite clay in the adsorption of viruses (Sobsey, M. D. and Cromeans, T., 1985; Lipson, S. M. & Stotzky, G., 1985), for example, have been studied extensively in the last few decades due to its use in microbial filtration in the treatment of water.

Further, in the past century we have witnessed three pandemics of influenza, of which the “Spanish flu” of 1918 was the largest pandemic of any infectious disease known to medical science (Oxford, J. S., 2000). The three strains which caused these pandemics belong to group A of the influenza viruses and, unlike the other two groups (B and C); this group infects a vast variety of animals (poultry, swine, horses, humans and other mammals).

Influenza A viruses continue to cause global problems, both economically and medically (Hayden, F. G. & Palese, P., 2000). The recent South East Asian outbreaks of avian influenza in 2003 and 2004 are ideal examples of this.

Much has been done to control and prevent another pandemic from occurring with many anti-influenza products (vaccines and treatments) currently on the market. The most recognized of these is TAMIFLU® (oseltamivir phosphate), a neuraminidase inhibitor, which functions by preventing spread of the virus within the human body.

Scientists have, in the recent years, been looking to develop new drugs following novel strategies of coping with Influenza. With the numbers of such projects on the rise researchers have been focusing on different Influenza target sites in which to develop new vaccines and treatments. Fiers, W. et al. (2004), for example, have reported the efficacy of an M2e vaccine, which targets the less variable M2 transmembrane protein of the influenza virus. Another example is the “OX40 treatment”, which reduces the excessive immune response that accompanies Influenza infections and which can increase the severity of symptoms (Hussell, T. et al. (2004).

Acute viral gastroenteritis is a very common illness which occurs in both epidemic and endemic forms. It affects all age groups worldwide and also includes some of the commonly encountered traveler's diarrhea. This syndrome is recognized as being second in frequency only to the common cold among illnesses affecting U.S. families under epidemiological surveillance. The clinical presentation of the illness is variable, but in general it is self-limited, has an explosive onset, and is manifested by varying combinations of diarrhea, nausea, vomiting, low-grade fever, abdominal cramps, headache, anorexia, myalgia, and malaise. It is not only responsible for a great deal of misery and time lost from school and work, but can be severe, indeed fatal, in the infant, elderly, or debilitated patient. Due to associated malabsorption, viral gastroenteritis may trigger or enhance the morbidity associated with malnutrition in marginally nourished populations. See Cukor et al., (Microbiol. Rev., 48:157-179, 1984) for further review of viral gastroenteritis.

The recent outbreaks of foot-and-mouth disease (FMD) in a number of FMD-free countries, in particular Taiwan in 1997 and the United Kingdom in 2001, have significantly increased public awareness of this highly infectious disease of cloven-hoofed livestock. Outbreaks have occurred in every livestock-containing region of the world with the exception of New Zealand, and the disease is currently enzootic in all continents except Australia and North America. The disease affects domestic cloven-hoofed animals, including cattle, swine, sheep, and goats, as well as more than seventy species of wild animals, including deer (Fenner et al., Veterinary Virology, p. 403-430, 1993), and is characterized by fever, lameness, and vesicular lesions on the tongue, feet, snout, and teats. Other vesicular diseases, such as swine vesicular disease (SVD), vesicular stomatitis, and vesicular exanthema of swine, cause signs so similar to those of FMD that differential clinical diagnosis alone can be difficult (Bachrach et al., Annu. Rev. Microbiol. 22:201-244, 1968). Although FMD does not result in high mortality in adult animals, the disease has debilitating effects, including weight loss, decrease in milk production, and loss of draught power, resulting in a loss in productivity for a considerable time. Mortality, however, can be high in young animals, where the virus can affect the heart. In addition, cattle, sheep, and goats can become carriers, and cattle can harbor virus for up to 2 to 3 years (Brooksby et al., Intervirology, 18:1-23, 1982). See Grubman et al., (Clin. Microbiol. Rev., 17:465-493) for a further review of foot and mouth disease.

Layered phyllosilicates, such as bentonite clay, or montmorillonite clay, are the active virus-interacting minerals described herein for inactivating viruses. Their virus sorption/binding properties, in prior art theory, are due to their negative electrical charge, which attracts positively charged toxins (including bacteria and viruses) and binds them. The virucidal phyllosilicates described herein, however, bind both positively charged and negatively charged virus molecules. It is theorized that sorption and/or binding of the virus to the layered phyllosilicates described herein is achieved by one or more mechanisms selected from the group consisting of adsorption; ionic complexing; electrostatic complexing; chelation; hydrogen bonding; ion-dipole; dipole/dipole; Van Der Waals forces; and any combination thereof. Such ionic bonding, e.g., via one or more cations or negative charge sites of the phyllosilicate sharing electrons with one or two atoms of one or two polar ends of a virus molecule, on a phyllosilicate surface, provides inactivation of a surprisingly high percentage of the virus molecules.

### SUMMARY OF THE INVENTION

It has been found that layered phyllosilicates are useful for adsorbing and/or binding to and, thereby, inactivating viruses, particularly both the human immunodeficiency virus (HIV) and Influenza A virus. The ability of a layered phyllosilicate to interact with and inactivate two very different acting viruses is most unexpected.

The layered phyllosilicate material useful for virus interaction, as described herein, includes the following clay minerals: montmorillonite, particularly sodium montmorillonite, protonated hydrogen montmorllonite, magnesium montmorillonite and/or calcium montmorillonite; nontronite; beidellite; laponite; yakhontovite; zincsilite; volkonskoite; hectorite; saponite; ferrosaponite; sauconite; swinefordite; pimelite; sobockite; stevensite; svinfordite; venniculite; synthetic clays; mixed layered illite/smectite minerals, such as rectorite, tarosovite, and ledikite; admixtures of illites with the clay minerals named above, and the magnesium aluminum silicates. Any one or any mixture of two or more of the above clay minerals is capable of adsorbing, and/or ionically bonding with, any virus, or combination of viruses, thereby inactivating the virus (es).

One preferred layered phyllosilicate is a smectite clay having at least 80%, preferably at least 95% interlayer, exchangeable homoionic cations, preferably sodium ions, based on the total of number of interlayer, exchangeable cations. Other particularly effective phyllosilicates that are effective in interacting with and inactivating significant percentages of a host of viruses, particularly HIV and influenza A viruses, include protonated onium ion-exchanged layered phyllosilicates (protonated organoclays); smectite clays having a particle size less than 74 μm, preferably less than 50 μm, more preferably less than 20 μm; and exfoliated smectite clays, including individual clay platelets and tactoids of 5 or less platelet layers.

In accordance with one embodiment for using the virucidal layered phyllosilicates described herein, the phyllosilicate particles are sprayed onto an absorbent mask as an air purification device, or included in a hand wipe material (hand sanitizers) for cleaning virus-contaminated surfaces, thereby adsorbing and inactivating the viruses, thereby preventing viruses from being breathed into the nose and mouth of a person or for adsorbing and thereby inactivating viruses from the hands, e.g., before handling a baby; or on gloves to inactivate viruses.

In other embodiments, the virucidal layered phyllosilicates can be suspended in lotions, shampoos and foams or skin creams, gels and ointments that are applied to skin, particularly hands and face, or internally within the vagina, for interacting with and thereby inactivating the transfer of viruses from one person to another, or to prevent a person from transferring the virus from external skin to internal cells.

In still another embodiment, the virucidal layered phyllosilicates can be ingested in the form of powder or liquid solution or suspension which can further be filled into a capsule or compressed into a tablet for internal interaction and inactivation of viruses within the gastrointestinal tract that have been or are about to be ingested. When wastes are expelled, viruses are retained inactivated on the clay and prevented from causing secondary infections.

In another embodiment, the virucidal layered phyllosilicates can be vaginally inserted for interaction and inactivation of HIV or other sexually-transmitted viruses, in the same manner as a spermicidal foam or body heat-dissolving spermicidal cartridge.

In still another embodiment, the virucidal layered phyllosilicates can be held in a vessel for filtering contact with blood, e.g., a secondary dialysis filter, or for filtering viruses from water in a virus-removing water purification step or during processing and manufacturing of biopharmaceuticals, such as monoclonal antibodies and vaccines.

In another embodiment, the virucidal layered phyllosilicates can be used as, or form a portion of, a HVAC filtration media to prevent virus-contaminated air from passing between rooms, for example, between rooms in a hospital.

In another embodiment, the virucidal layered phyllosilicates are used as a nasal spray or mucoadhesive gel or paste within the nasal cavity by spraying a suspension of the virucidal phyllosilicate in a suitable biocompatible carrier (including water and/or organic solvent) into the nasal passages to coat nasal cells. In this manner, viruses entering the nose will interact with the phyllosilicate and thereby will be inactivated to prevent infection.

In still another embodiment, a condom is coated with a suspension of the virucidal layered phyllosilicates, in a cosmetically acceptable carrier, e.g., water and/or solvent. In the event of condom failure, the virucidal phyllosilicate interacts with and inactivates viruses before a sexual partner is infected.

In another embodiment, a suspension of the virucidal layered phyllosilicate in a cosmetically acceptable carrier is packaged in a portable container, e.g., a tube or bottle, for use on the hands to periodically inactivate viruses held on a person's skin.

In another embodiment, the virucidal layered phyllosilicates can be dispensed throughout a virus-contaminated body of water, such as a pond or lake, to inactivate viruses therein.

The virucidal layered phyllosilicates described herein interact with viruses, adsorb and/or bind them ionically to the virucidal layered phyllosilicates, thereby preventing the viruses from migrating to and penetrating cell membranes, thereby preventing the viruses from reproducing and rupturing the cells and releasing more of the virus attaching to and infecting host cells.

Also provided is a method of inactivating a virus in the gastrointestinal tract of a mammalian subject comprising administering to said subject a composition comprising a layered phyllosilicate material in an amount effective for virus inactivation. A method of treating a viral infection in the gastrointestinal tract of a mammalian subject comprising administering to said subject a composition comprising a layered phyllosilicate material and a pharmaceutically acceptable carrier is also provided.

In some aspects, the composition further comprises a pharmaceutically acceptable carrier, diluent or adjuvant. In some aspects, mammalian subject is a human. In other aspects, the mammalian subject is animal selected from the group consisting of a horse, a cow, sheep, a pig, a llama, an alpaca, a goat, a dog, a cat, a mouse, a rat, a rabbit, a guinea pig and a hamster.

In one aspect, the layered phyllosilicate material comprises at least 90% homoionic interlayer exchangeable cations, in relation to all interlayer exchangeable cations, and has a particle size less than 74 μm. In another aspect, the phyllosilicate material comprises interlayer exchangeable cations that are predominantly hydrogen cations. In another aspect, the layered phyllosilicate material comprises exfoliated platelets and/or tactoids of the layered phyllosilicate material.

In one embodiment, the virus in the gastrointestinal tract of the mammalian subject is an enterovirus; selected from the group consisting of polioviruses, coxsackieviruses, and echoviruses or is a virus is from a genus selected from the group consisting of calciviridae, norovirus and reoviridae. In another embodiment, the virus is norovirus, feline calcivirus or rotavirus.

In another embodiment, the invention provides a method of delivering a therapeutic agent to a mammalian subject in need thereof comprising administering a composition comprising a therapeutic agent and a layered phyllosilicate material. In one aspect, the therapeutic agent is selected from the group consisting of a nucleic acid, a protein, and a small molecule drug and is intercalated within the layered phyllosilicate material. In another aspect, the therapeutic agent is selected from the group consisting of colloidal silver, an antisense nucleotide, a thrombin inhibitor, an antithrombogenic agent, a tissue plasminogen activator, a thrombolytic agent, a fibrinolytic agent, a vasospasm inhibitor, a calcium channel blocker, a nitrate, a nitric oxide promoter, a vasodilator, an antimicrobial agent, an antibiotic, an antiplatelet agent, an antimitotic, a microtubule inhibitor, an actin inhibitor, a remodeling inhibitor, an agent for molecular genetic intervention, a cell cycle inhibitor, an inhibitor of the surface glycoprotein receptor, an antimetabolite, an antiproliferative agent, an anti-cancer chemotherapeutic agent, an anti-inflammatory steroid, an immunosuppressive agent, an antibiotic, a radiotherapeutic agent, iodine-containing compounds, barium-containing compounds, a heavy metal functioning as a radiopaque agent, a peptide, a protein, an enzyme, an extracellular matrix component, a cellular component, a biologic agent, an angiotensin converting enzyme (ACE) inhibitor, ascorbic acid, a free radical scavenger, an iron chelator, and an antioxidant.

In another embodiment, the invention provides a patch comprising a pad material having an upper surface and lower surface, an adhesive on the lower surface, and a therapeutic composition, wherein the therapeutic composition comprises a layered phyllosilicate material.

In yet another embodiment, the invention provides a surgical suturing thread coated or impregnated with a composition, wherein said composition comprises a layered phyllosilicate material.

In another embodiment, the invention provides a method of promoting the absorption of a therapeutic agent through the mucosal membranes in a mammalian subject, comprising administering to said subject a composition comprising a therapeutic agent, a layered phyllosilicate material and pharmaceutically acceptable carrier, diluent or excipient.

In yet another embodiment, the invention provides a method of delivering a diagnostic agent to a biological fluid or a subject comprising administering a composition comprising a diagnostic agent and a layered phyllosilicate material.

In yet another embodiment, the invention provides a method of inactivating a virus in waste expelled from a mammal comprising administering to said mammal a composition comprising a layered phyllosilicate material and pharmaceutically acceptable carrier, diluent or excipient in an amount effective for virus inactivation. In some aspects, the waste is fecal matter. In other aspects, the waste is urine.

Whenever used in this specification, the terms set forth shall have the following meanings:

Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the terms “antiviral” or “antiviral activity” refers to the ability of the composition, method, or treatment regimen to reduce the size, extent, severity, and duration of infections, or the communicability of the virus.

As used herein, the terms “therapeutically effective” or “amount sufficient” refers to when a composition or method of the invention is properly administered in vivo to a vertebrate, such as a bird or mammal, including humans, a measurable beneficial effect occurs. Exemplary beneficial effects include measurable antiviral effects in conditions where viral load can be assayed; a reduction of clinically verifiable and/or patient-reported symptoms or complete resolution or curing of the viral infection or outbreak or other diseases.

As used herein, the term “virucidal” means capable of inactivating or destroying a virus.

“Phyllosilicate” or “Virucidal Clay” shall mean clay minerals, e.g., montmorillonite, particularly sodium montmorillonite, magnesium montmorillonite and/or calcium montmorillonite; protonated montmorillonite nontronite; beidellite; laponite; yakhontovite; zincsilite; volkonskoite; hectorite; saponite; ferrosaponite; sauconite; swinefordite; pimelite; sobockite; stevensite; svinfordite; vermiculite; synthetic clays; mixed layered illite/smectite minerals, such as rectorite, tarosovite, and ledikite; admixtures of illites with the clay minerals named above, and the magnesium aluminum silicates.

“Homoionic Phyllosilicate” shall mean a layered Phyllosilicate material that has been purified by ion-exchange, for example, as described in this assignee's U.S. Pat. No. 6,050,509, to contain at least 90% of a single element, in relation to all interlayer exchangeable cations, from periodic table groups 1a, 2a, 3b, 4b, 5b, 6b, 7b, 8, 1b, 2b, 3a, tin and lead; or a protonated onium ion compound, as the interlayer exchangeable cations.

“Platelets” shall mean individual layers of a Phyllosilicate.

“Intercalate” or “Intercalated” shall mean a phyllosilicate material that includes an onium ion spacing agent, preferably a protonated onium ion spacing agent, disposed between adjacent platelets of the layered Phyllosilicate material to increase the interlayer spacing between the adjacent platelets by at least 3 Å, preferably at least 5 Å, to an interlayer spacing, for example, of at least about 8 Å, preferably at least about 10 Å.

“Intercalation” shall mean a process for forming an Intercalate.

“Onium Ion Intercalant” or “Onium Ion Spacing Agent” or “Onium Ion Compound” shall mean an organic compound, preferably a protonated organic compound, that includes at least one positively charged atom selected from the group consisting of a nitrogen atom, a phosphorous atom, a sulfur atom or an oxygen atom, preferably a quaternary ammonium compound, and when dissolved in water and/or an organic solvent, an anion dissociates from the onium ion spacing agent leaving an onium cation that can ion-exchange with a silicate platelet exchangeable cation of the Phyllosilicate, e.g., Na⁺, Ca⁺², Li⁺, Mg⁺², Al⁺³, or K⁺.

“Intercalating Carrier” shall mean a carrier comprising water and/or an organic liquid to form an Intercalating Composition capable of achieving Intercalation of an onium ion spacing agent which ion-exchanges with exchangeable interlayer cations of the layered Phyllosilicate.

“Intercalating Composition” shall mean a composition comprising one or more onium ion spacing agents, an Intercalating Carrier for the onium ion spacing agent, and a layered Phyllosilicate.

“Exfoliate” or “Exfoliated” shall mean individual platelets of an Intercalated layered Phyllosilicate so that adjacent platelets of the Intercalated layered Phyllosilicate can be dispersed individually throughout a carrier material, such as water, a polymer, an alcohol or glycol, or any other organic liquid, together with tactoids of 2-20 layers of non-exfoliated platelets.

Exfoliation” shall mean a process for forming an Exfoliate from an Intercalate.

### Clay Purification and Ion-Exchange

A preferred layered phyllosilicate useful for interaction with an inactivation of viruses is a smectite clay that has been purified and ion-exchanged in accordance with this assignee's U.S. Pat. No. 6,050,509, hereby incorporated by reference. The ion-exchange process can be used to provide a homoionic layered phyllosilicate or can be used to provide the phyllosilicate with mixed cations from the periodic table groups 1a, 1b, 2a, 2b, 3a, 3b, 4b, 5b, 6b, 7b, 8, tin, hydrogen, lead, and/or protonated onium ions, within any percentage of the phyllosilicate exchangeable cations (1-99% of the exchangeable cations). According to U.S. Pat. No. 6,050,509 the smectite clay slurry is pumped to a series of ion exchange columns where any undesirable cation is exchanged with a desirable cation. In this manner, the crude montmorillonite clay can be exchanged to produce a purified montmorillonite with a single (homoionic) desirable cation or with a mixture of cations. In this manner, by using the appropriate ion exchange column, any element can be exchanged for the interlayer cations of a phyllosilicate for virus inactivation, including hydrogen and/or one or more elements from the following groups of the periodic table: group 1a (e.g., lithium, sodium, potassium) group 2a (e.g., magnesium, calcium, barium) group 3b (e.g., lanthanium), group 4b (e.g., titanium) group 5b (e.g., vanadium), group 6b (e.g., chromium), group 7b (e.g., manganese) group 8 (e.g., iron, cobalt, nickel, platinum), group 1b (e.g., copper, gold, silver), group 2b (e.g., zinc, cadmium) group 3a (e.g., boron, aluminum) and selected members of group 4a (e.g., tin and lead). In this manner, one could exchange a metal or metal cation with known, good antimicrobial or antiviral properties on the surface of the montmorillonite clay, or any layered phyllosilicate material, to produce a material with superior antimicrobial and antiviral properties. Homoionic hydrogen ion-exchanged layered phyllosilicates are formed as follows: (1) A slurry of 1% by weight of sodium montmorillonite clay in de-ionized water was prepared; (2) The 1% by weight sodium montmorillonite slurry was pumped through an ion-exchange column filled with hydrogen ion-exchange beads. The hydrogen ion-exchange beads were formed by contacting ion-exchange beads with an excess of 2N HCl; and (3) the hydrogen ion-exchanged slurry was diluted to 0.1% by weight for testing.

In accordance with this embodiment of the virucidal layered phyllosilicate, the crude layered phyllosilicate deposits initially include one or more of the following non-smectite impurities: (SiO₂), feldspar (KAlSi₃O₈), opal-CT (SiO₂); gypsum (CaSO₄.2H₂O); albite (NaAlSi₃O₈); anorthite (CaAl₁₂Si₂O₈); orthoclase (KAlSi₃O₈); apatite (Ca₅ (PO₄)₃(F,Cl,OH)); halite (NaCl); calcite (CaCO₃); dolomite (CaMg(CO₃)₂; sodium carbonate (Na₂CO₃); siderite (FeCO₃) biotite (K(Mg,Fe)₃(AlSi₃O₁₀) (OH)₂) muscovite (KAl₂(AlSi₃O₁₀) (OH)₂); chlorite ((Mg,Fe)₆(Si,Al)₄O₁₀ (OH)₈); stilbite (NaCa₂Al₅Si₁₃O₃₆.14H₂O); pyrite (FeS₂); kaolinite (Al₂Si₂O₅.(OH)₄); and hematite (Fe₂O₃)

In order to remove at least 90% by weight of the above impurities, preferably at least 99% of the impurities, preferably, the layered phyllosilicate is dispersed in water, preferably at a concentration of about 10% to about 15% by weight, based on the total weight of phyllosilicate and water. The preferred layered phyllosilicate is a smectite clay, such as a montmorillonite clay, that is predominantly (greater than about 50% by weight) sodium or calcium montmorillonite clay so that the concentration of clay dispersed in water can be as high as about 15% by weight. If, for example, a sodium montmorillonite clay is dispersed in water, the higher swelling capacity of sodium montmorillonite in water will result in a viscosity that is too high for handling at a concentration of about 6-10% by weight. Accordingly, in order to achieve the most efficient purification of the smectite clay, it is preferred that the clay dispersed in water is a montmorillonite clay having predominantly (at least 50% by number) multivalent cations, i.e., Ca⁺² in the interlayer space, such as calcium montmorillonite clay. If the clay is not predominantly a multivalent clay, such as calcium montmorillonite, it can be ion-exchanged sufficiently to provide predominantly multivalent ions in the interlayer spaces between montmorillonite clay platelets.

The clay slurry is then directed into a series of cascaded hydrocyclones of decreasing size, each hydrocyclone capable of removing impurities of at least a particular size, particularly the impurities having a size greater than about 74 microns. The resulting clay, separated from the impurities, has a particle size such that at least about 90% by volume of the clay particles have a size below about 74 microns, preferably below about 50 microns, more preferably below about 20 microns. The clay slurry is then directed upwardly through a cation exchange column that removes multivalent interlayer cations from the montmorillonite clay (e.g., divalent and/or trivalent cations) and substitutes monovalent cations such as sodium, lithium and/or hydrogen for the multivalent cations within the interlayer spaces between platelets of the montmorillonite clay.

After essentially complete ion exchange, such that the clay has at least 90%, preferably at least 95%, more preferably at least 99%, by number, monovalent cations in the interlayer spaces, the clay preferably is then directed into a high speed centrifuge where the clay is subjected to centrifugal force equal to, for example, at least about 2,000 G (forces of gravity) up to about 4,000 G, preferably about 2,500 G to about 3,500 G, capable of removing clay particle sizes between about 5 microns and about 74 microns, such that the remaining montmorillonite clay particles, having less than about 50 by weight crystalline and amorphous non-smectite clay impurities, preferably less than about 5% by weight impurities therein, have a particle size of about 10 microns or less, preferably about 8 microns or less, and have an average particle size less than about 3 microns, preferably less than about 2 microns.

In accordance with an important feature of this embodiment, for effective removal of the impurities that have a size less than about 10 microns in diameter, the clay should first be conditioned or treated for removal of all multivalent, e.g., divalent and trivalent, interlayer cations by substitution of the multivalent cations with one or more monovalent cations, such as sodium ions, or protonated onium ions, in order to provide effective removal of the smallest impurities, for example, in a high speed (2,000-4,000 G) centrifuge. In accordance with another important feature of this embodiment, it has been found that conveying the clay slurry through the hydrocyclones prior to monovalent, e.g., sodium ion-exchange provides for a much more efficient process since the material fed to the hydrocyclones can be fed at a higher solids content without an undue increase in the viscosity of the material fed to the hydrocyclones. Accordingly, ion-exchange is accomplished after the clay slurry is passed through the hydrocyclones and before sending the partially purified clay slurry to a centrifuge for removal of the smallest impurities removed from the product.

The product from primary and secondary one inch hydrocyclones are fed by gravity to an ion-exchange feed tank where the clay/water slurry, including impurities, are maintained at a clay concentration of about 1-7% by weight, preferably about 3-7% by weight, based on the total weight of material in the ion-exchange feed tank. The clay slurry from the ion-exchange feed tank is pumped to a series of ion-exchange columns where the interlayer clay cations are exchanged with cations from periodic table groups 1a, 1b, 2a, 2b, 3a, 3b, 4b, 5b, 6b, 7b, 8, tin or lead, preferably sodium. Ion-exchange is achieved, for example, by contact with an ion-exchange resin, preferably PUROLITE C-100, obtained from The PUROLITE Company, a polystyrene cross linked with divinyl benzene, in spherical bead form, in the sodium ionic form, having an 8% by weight divinyl benzene content.

The product from a secondary one inch hydrocyclone includes at least about 90% by number particles having a size less than about 50 microns, preferably less than about 20 microns, more preferably less than about 10 microns, a mean particle size less than about 10 microns, and a median particle size less than about 5 microns.

### Exfoliated Clay to Form Clay Platelets and/or Tactoids

To form the intercalated and exfoliated layered phyllosilicates described herein, the phyllosilicate material, e.g., bentonite, should be swelled or intercalated, in the preferred embodiment, by sorption of an onium ion spacing agent.

While the compositions and methods described herein are described by way of the preferred embodiment via expanding the interlaminar spacing between adjacent platelets of a layered phyllosilicate material by intercalating onium ions between the silicate platelets, the interlaminar spacing also can be achieved by intercalating a silane coupling agent, or by an acidification technique, by substitution with hydrogen (ion-exchanging the interlayer cations with hydrogen by use of an acid or ion-exchange resin) as disclosed in the Deguchi U.S. Pat. No. 5,102,948, and in the Lan, et al. U.S. Pat. No. 5,853,886, both patents hereby incorporated by reference. In this clay exfoliation embodiment, the extremely small size of the individual platelets and clay tactoids should permit interaction with and inactivation of all viruses, including neoviruses, polioviruses type 2, enteroviruses, bovine rotavirus, and bovine corona viruses.

Sorption of the onium ion spacing agent should be sufficient to achieve expansion of the interlayer spacing of adjacent platelets of the layered phyllosilicate material (when measured dry) by at least about 3 Å, preferably at least about 5 Å.

The onium ion spacing agent is introduced into the layered phyllosilicate galleries in the form of a solid or liquid composition (neat or aqueous, with or without an organic solvent, e.g., an aliphatic hydrocarbon, such as heptane to, if necessary, aid to dissolve the onium ion compound) having an onium ion spacing agent concentration sufficient to provide a concentration of about 5% to about 10% by weight phyllosilicate (90-95% water) and the onium ion compound is dissolved in the phyllosilicate slurry water, preferably at a molar ratio of onium ions to exchangeable interlayer cations of at least about 0.25:1, more preferably at least about 0.5:1, most preferably at least about 1:1. The onium ion-intercalated layered phyllosilicate then is separated from the water easily, since the phyllosilicate is now hydrophobic, and dried in an oven to less than about 15% water, preferably bone dry, before interaction with the virus. The onium ion spacing agent compound can be added as a solid with the addition to the layered phyllosilicate material/onium ion compound blend of preferably at least about 20% water, more preferably at least about 30% water or more, based on the dry weight of layered material. Preferably about 30% to about 50% water, more preferably about 30% to about 40% water, based on the dry weight of the layered material, is included in the onium ion intercalating composition, so that less water is sorbed by the intercalate, thereby necessitating less drying energy after onium ion intercalation.

The onium ion spacing agent cations intercalated via ion-exchange into the interlayer spaces between adjacent layered material platelets are primary, secondary, tertiary or quaternary onium ions having the following preferred structure: 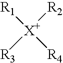
wherein X═N, P, S, or O; and
wherein R₁, R₂, R₃ and R₄ are H or organic moieties, such as linear or branched alkyl, aryl or aralkyl moieties having 1 to about 24 carbon atoms.

The more preferred protonated C₆+ onium ions are preferably quaternary ammonium ions having Formula 1, as follows: 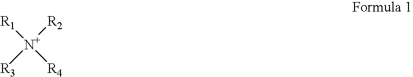
wherein R₁ is a long chain alkyl moiety ranging from C₆ to C₂₄, straight or branched chain, including mixtures of long chain moieties, i.e., C₆, C₈, C₁₀, C₁₂, C₁₄, C₁₆, C₁₈, C₂₀, C₂₂ and C₂₄, alone or in any combination; and R₂, R₃ and R₄ are moieties, same or different, selected from the group consisting of H, alkyl, benzyl, substituted benzyl, e.g., straight or branched chain alkyl-substituted and halogen-substituted; ethoxylated or propoxylated alkyl; ethoxylated or propoxylated benzyl, e.g., 1-10 moles of ethoxylation or 1-10 moles of propoxylation. Preferred protonated onium ions include protonated octadecylamine, protonated hexyl amine; protonated octyl amine; protonated tallow amine; protonated tallow diamine; protonated tallow triamine; protonated tallow tetraamine; protonated hydrogenated tallow amine; protonated hydrogenated tallow diamine; protonated hydrogenated tallow triamine; protonated hydrogenated tallow tetraamine; protonated octadecyl amine; and mixtures thereof.
R¹—X⁺—R—Y⁺
where X⁺ and Y⁺, same or different, are ammonium, sulfonium, phosphonium, or oxonium radicals such as ⁺NH₃, ⁺NH₂—, ⁺N(CH₃)₃, ⁺N(CH₃)₂—, ⁺N(CH₃)₂(CH₂CH₃), ⁺N(CH₃)(CH₂CH₃)—, ⁺S(CH₃)₃, ⁺S(CH₃)₂—, ⁺P(CH₃)₃, ⁺P(CH₃)₂—, ⁺NH₄, ⁺NH₃—, and the like; R is an organic spacing, backbone radical, straight or branched, preferably having from 2 to 24, more preferably 3 to 10 carbon atoms, in a backbone organic spacing molecule covalently bonded at its ends to charged N⁺, P⁺, S⁺ and/or O⁺ cations and R¹ can be hydrogen, or an alkyl radical of 1 to 22 carbon atoms, linear or branched, preferably having at least 6 carbon atoms. Examples of R include substituted or unsubstituted alkylene, cycloalkenylene, cycloalkylene, arylene, alkylarylene, either unsubstituted or substituted with amino, alkylamino, dialkylamino, nitro, azido, alkenyl, alkoxy, cycloalkyl, cycloalkenyl, alkanoyl, alkylthio, alkyl, aryloxy, arylalkylamino, alkylamino, arylamino, dialkylamino, diarylamino, aryl, alkylsufinyl, aryloxy, alkylsulfinyl, alkylsulfonyl, arylthio, arylsulfinyl, alkoxycarbonyl, arylsulfonyl, or alkylsilane. Examples of R1 include non-existent; H; alkyl having 1 to 22 carbon atoms, straight chain or branched; cycloalkenyl; cycloalkyl; aryl; alkylaryl, either unsubstituted or substituted or substituted with amino, alkylamino, dialkylamino, nitro, azido, alkenyl, alkoxy, cycloalkyl, cycloalkenyl, alkanoyl, alkylthio, alkyl, aryloxy, arylalkylamino, alkylamino, arylamino, dialkylamino, diarylamino, aryl, alkylsufinyl, aryloxy, alkylsulfinyl, alkylsulfonyl, arylthio, arylsulfinyl, alkoxycarbonyl, arylsulfonyl, or alkylsilane. Illustrative of useful R groups are alkylenes, such as methylene, ethylene, octylene, nonylene, tert-butylene, neopentylene, isopropylene, sec-butylene, dodecylene and the like; alkenylenes such as 1-propenylene, 1-butenylene, 1-pentenylene, 1-hexenylene, 1-heptenylene, 1-octenylene and the like; cycloalkenylenes such as cyclohexenylene, cyclopentenylene and the like; alkanoylalkylenes such as butanoyl octadecylene, pentanoyl nonadecylene, octanoyl pentadecylene, ethanoyl undecylene, propanoyl hexadecylene and the like; alkylaminoalkylenes, such as methylamino octadecylene, ethylamino pentadecylene, butylamino nonadecylene and the like; dialkylaminoalkylene, such as dimethylamino octadecylene, methylethylamino nonadecylene and the like; arylaminoalkylenes such as phenylamino octadecylene, p-methylphenylamino nonadecylene and the like; diarylaminoalkylenes, such as diphenylamino pentadecylene, p-nitrophenyl-p-α-methylphenylamino octadecylene and the like; alkylarylaminoalkylenes, such as 2-phenyl-4-methylamino pentadecylene and the like; alkylsulfinylenes, alkylsulfonylenes, alkylthio, arylthio, arylsulfinylenes, and arylsulfonylenes such as butylthio octadecylene, neopentylthio pentadecylene, methylsulfinyl nonadecylene, benzylsulfinyl pentadecylene, phenylsulfinyl octadecylene, propylthiooctadecylene, octylthio pentadecylene, nonylsulfonyl nonadecylene, octylsulfonyl hexadecylene, methylthio nonadecylene, isopropylthio octadecylene, phenylsulfonyl pentadecylene, methylsulfonyl nonadecylene, nonylthio pentadecylene, phenyltlio octadecylene, ethyltio nonadecylene, benzylthio undecylene, phenethylthio pentadecylene, sec-butylthio octadecylene, naphthylthio undecylene and the like; alkoxycarbonylalkylenes such as methoxycarbonylene, ethoxycarbonylene, butoxycarbonylene and the like; cycloalkylenes such as cyclohexylene, cyclopentylene, cyclo-octylene, cycloheptylene and the like; alkoxyalkylenes such as methoxy-methylene, ethoxymethylene, butoxymethylene, propoxyethylene, pentoxybutylene and the like; aryloxyalkylenes and aryloxyarylenes such as phenoxyphenylene, phenoxymethylene and the like; aryloryalkylenes such as phenoxydecylene, phenoxyoctylene and the like; arylalkylenes such as benzylene, phenthylene, 8-phenyloctylene, 10-phenyldecylene and the like; alkylarylenes such as 3-decylphenylene, 4-octylphenylene, 4-nonylphenylene and the like; and polypropylene glycol and polyethylene glycol substituents such as ethylene, propylene, butylene, phenylene, benzylene, tolylene, p-styrylene, p-phenylmethylene, octylene, dodecylene, octadecylene, methoxy-ethylene, moieties of the formula —C₃H₆COO—, —C₅H₁₀COO—, —C₇H₁₀COO—, —C₇H₁₄COO—, —C₉H₁₈COO—, —C₁₁H₂₂COO—, —C₁₃H₂₆COO—, —C₁₅H₃₀COO—, and —C₁₇H₃₄COO— and —C═C(CH₃)COOCH₂CH₂—, and the like. Such tetra-, tri-, and di-ammonium, -sulfonium, -phosphonium, -oxonium; ammonium/sulfonium; ammonium/phosphonium; ammonium/oxonium; phosphonium/oxonium; sulfonium/oxonium; and sulfonium/phosphonium radicals are well known in the art and can be derived from the corresponding amines, phosphines, alcohols or ethers, and sulfides.

Other useful spacing agent compounds are multi-onium ion compounds that include at least two primary, secondary, tertiary or quaternary ammonium, phosphonium, sulfonium, and/or oxonium ions having Formula 2, as follows: 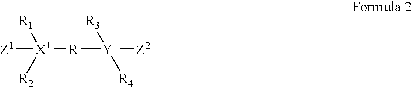
wherein R is an alkylene, aralkylene or substituted alkylene charged atom spacing moiety, preferably ranging from C₃ to C₂₄, more preferably about C₃ to C₆ for relatively high charge density (150 milliequivalents/100 grams C.E.C. to 70 milliequivalents/100 grams C.E.C.) layered materials; and preferably from C₆ to C₁₂ for medium to low charge density (70 milliequivalents/100 grams C.E.C. to 30 milliequivalents/100 grams C.E.C.) layered materials. R can be straight or branched chain, including mixtures of such moieties, i.e., C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃ and C₂₄, alone or in any combination; and R₁, R₂, R₃ and R₄ are moieties, same or different, selected from the group consisting of hydrogen, alkyl, aralkyl, benzyl, substituted benzyl, e.g., straight or branched chain alkyl-substituted and halogen-substituted; ethoxylated or propoxylated alkyl; ethoxylated or propoxylated benzyl, e.g., 1-10 moles of ethoxylation or 1-10 moles of propoxylation. Z¹ and Z², same or different, may be non-existent, or may be any of the moieties described for R₁, R₂, R₃ or R₄. Also, one or both of Z¹ and Z² may include one or more positively charged atoms or onium ion molecules.

Any swellable layered phyllosilicate material that sufficiently sorbs the onium ion spacing agent to increase the interlayer spacing between adjacent phyllosilicate platelets by at least about 3 Å, preferably at least about 5 Å, can be used in the practice of this invention. Useful swellable layered materials include phyllosilicates, such as smectite clay minerals, e.g., montmorillonite, particularly sodium montmorillonite, magnesium montmorillonite and/or calcium montmorillonite; nontronite; beidellite; laponite; yakhontovite; zincsilite; volkonskoite; hectorite; saponite; ferrosaponite; sauconite; swinefordite; pimelite; sobockite; stevensite; svinfordite; vermiculite; synthetic clays; mixed layered illite/smectite minerals, such as rectorite, tarosovite, and ledikite; admixtures of illites with the clay minerals named above, magnesium aluminum silicates; ion-exchanged phyllosilicates, including homoionic and/or protonated phyllosilicates; and mixtures of any two or more of the above-listed phyllosilicates. Exemplary mixtures include any of the above-listed phyllosilicates, wherein one of the above-listed phyllosilicates is present in amount ranging from about 1%-99% wt. and another phyllosilicate is present in an amount ranging from 99%-1% wt.; or wherein one of the above-listed phyllosilicates is present in amount greater than 50% wt and another phyllosilicate is present in an amount less than 50% wt; or wherein one of the above-listed phyllosilicates is present in amount of 50% wt and a second phyllosilicate is present in an amount of 50%; or wherein one of the above-listed phyllosilicates is present in amount of about 10% wt and another phyllosilicate is present in an amount of about 90%; or wherein one of the above-listed phyllosilicates is present in amount of about 20% wt and another phyllosilicate is present in an amount of about 80%; or wherein one of the above-listed phyllosilicates is present in amount of about 30% wt and another phyllosilicate is present in an amount of about 70% wt; or wherein one of the above-listed phyllosilicates is present in amount of about 40% wt and another phyllosilicate is present in an amount of about 60% wt. The weight percent indicated above is based on the weight of the clay mixture.

Preferred swellable layered materials are phyllosilicates of the 2:1 type having a negative charge on the layers ranging from about 0.15 to about 0.9 charges per formula unit and a commensurate number of exchangeable metal cations in the interlayer spaces. Most preferred layered materials are smectite clay minerals such as montmorillonite, nontronite, beidellite, volkonskoite, hectorite, saponite, sauconite, sobockite, stevensite, and svinfordite.

As used herein the “interlayer spacing” refers to the distance between the internal faces of the adjacent phyllosilicate layers as they are assembled in the layered material before any delamination (exfoliation) takes place. The preferred clay materials generally include interlayer cations such as Na⁺, Ca⁺², K⁺, Mg⁺², Al⁺³⁺, NH₄ and the like, including mixtures thereof, and can be ion-exchanged to include other cations such as the elements from period table group 1a, 1b, 2a, 2b, 3a, 3b, 4b, 5b, 6b, 7b, 8, tin and lead.

The onium ions, may be introduced into (sorbed within) the interlayer spaces of the layered phyllosilicate in a number of ways. In a preferred method of intercalating the onium ions between adjacent platelets of the layered material, the phyllosilicate material is slurried in water, e.g., at 5-20% by weight layered phyllosilicate material and 80-95% by weight water, and the onium ion compound is dissolved in the water in which the phyllosilicate material is slurried. If necessary, the onium ion compound can be dissolved first in an organic solvent, e.g., propanol. The phyllosilicate material then is separated from the slurry water and dried suspending the individual silicate platelets and tactoids in a liquid carrier.

To achieve sufficient intercalation of the onium ions between adjacent platelets of the layered phyllosilicate, the phyllosilicate/onium ion intercalating composition preferably contains a molar ratio of onium ions to layered phyllosilicate of at least 0.25:1, more preferably at least 0.5:1 for the onium ions to exchange interlayer cations with the smectite clay, most preferably 1:1, based on the dry weight of the phyllosilicate, so that the resulting onium ion-intercalated phyllosilicate has interior platelet surfaces that are sufficiently hydrophobic and sufficiently spaced for exfoliation and suspension of the individual platelets and tactoids in a liquid carrier. The onium ion carrier (preferably water, with or without an organic solvent) can be added by first solubilizing or dispersing the onium ion compound in the carrier; or a dry onium ion compound and relatively dry layered phyllosilicate (preferably containing at least about 4% by weight water) can be blended and the intercalating carrier added to the blend, or to the phyllosilicate prior to adding the dry onium ion. When intercalating the phyllosilicate with onium ions in slurry form, the amount of water can vary substantially, e.g., from about 4% by weight, preferably from a minimum of at least about 30% by weight water, with no upper limit to the amount of water in the intercalating composition (the phyllosilicate intercalate is easily separated from the intercalating composition due to its hydrophobicity after onium ion treatment).

Alternatively, the onium ion intercalating carrier, e.g., water, with or without an organic solvent, can be added directly to the phyllosilicate prior to adding the onium ion compound, either dry or in solution. Sorption of the onium ion compound molecules may be performed by exposing the phyllosilicate to a dry or liquid onium ion compound in the onium ion intercalating composition containing at least about 2% by weight, preferably at least about 5% by weight onium ion compound, more preferably at least about 10% onium ion compound, based on the dry weight of the layered phyllosilicate material.

In accordance with an emulsion method of intercalating the onium ions between the platelets of the layered phyllosilicate material, the phyllosilicate, preferably containing at least about 4% by weight water, more preferably about 10% to about 15% by weight water, is blended with water and/or organic solvent solution of an onium ion spacing agent compound in a ratio sufficient to provide at least about 5% by weight, preferably at least about 10% by weight onium ion compound, based on the dry weight of the layered phyllosilicate material.

The onium ion spacing agents have an affinity for the phyllosilicate so that they are sorbed between, and are ion-exchanged with the cations on the inner surfaces of the silicate platelets, in the interlayer spaces.

### PROTONATED ONIUM ION INTERCALATION EXAMPLES

### Example 1

Example 1 demonstrates the ion exchange process of smectite clay from a Ca form or Na/Ca mixed forms to Na-rich smectite clay.

Raw smectite clay was dispersed into water to make a 3 wt % clay slurry. This clay has a Na content of 0.20 wt % and Ca content of 2.10 wt %. The elemental analysis was measured by an X-ray fluorescence method. The mixture was mixed thoroughly with a mechanical mixer. The pH value of the starting clay slurry is 7-8. An ion exchange resin, such as Amberlite 200C Na, is available from Rohm & Hass packed in a glass column with a 2-in diameter and a 20-in length. A liquid pump was used to pump the clay slurry through the column at 20 ml/min. Elemental analysis of the finished clay, dried from the slurry, indicated that the Na content is 3.45 wt % and Ca content is 0.17 wt %. The ion exchanged clay is called E1-Na-Clay. This clay had a basal spacing of 13 Å.

### Example 2

Example 2 demonstrates the formation of protonated Octadecyl ammonium-treated smectite clay with Octadecyl ammonium acetate from the ion exchanged Na-smectite clay (E1-Na-clay) of Example 1.

100-g of sodium smectite clay E1-Na-clay was dispersed into 3000 ml water through a mechanical mixer. This clay slurry was heated to 80° C. 41.5 g of Octadecyl ammonium acetate from KAO Chemicals was added into the clay slurry. The clay showed excellent flocculation after the addition of the Octadecyl ammonium acetate. The pH of the clay reaction slurry was about 4. The clay was filtered with regular quantitative filter paper with the assistance of a mechanical vacuum pump. Then, the clay was dried in an oven over night at 80° C. and ground to pass through a 300-mesh screen as a fine powder. This modified clay was called E2-ODA-Clay.

### Example 3

Example 3 demonstrates the formation of protonated Octadecyl ammonium-treated smectite clay with a solution of Octadecyl ammonium ions in dilute HCl. (E3-ODA-Clay). This sample was measured by powder X-ray diffraction to determine the clay basal spacing after ion exchange. The result is listed in Table-1.

100-g of sodium smectite E1-Na-clay was dispersed into 3000 ml water through a mechanical mixer. This clay slurry was heated to 80° C. 33.8 g of Octadecyl amine was added into 1000 ml of 70° C. water and then mixed with 17.1 g of 10.5 N HCl. The Octadecyl amine-HCl solution was added into the clay slurry followed by mixing. The reaction slurry had a pH of 4. The clay showed excellent flocculation after the addition of the Octadecyl amine-HCl solution. The clay was filtered with regular quantitative filter paper with the assistance of a mechanical vacuum pump. Then, the clay was dried in an oven over night at 80° C. and ground to pass through a 300-mesh screen as a fine powder. This modified clay was called E3-ODA-Clay. This sample was measured by powder X-ray diffraction to determine the clay basal spacing after ion exchange. The result is listed in Table-1.

### Viruses and Viral Taxonomy

Viruses constitute a large and heterogeneous group, and they are classified in hierarchical taxonomic categories based on many different characteristics, e.g., morphology, antigenic properties, physiochemical and physical properties, proteins, lipids, carbohydrates, molecular properties, organization and replication, and biological properties. Whether the RNA or DNA is single or double stranded, the organization of the genome and the presence of particular genes comprise important aspects of the current taxonomy of viruses. All of the former are used to place a virus into a particular order or family. The classification is based upon macromolecules produced (structural proteins and enzymes), antigenic properties and biological properties (e.g., accumulation of virions in cells, infectivity, hemagglutination).

Viral classification is dynamic in that new viruses are continuously being discovered and more information is accumulating about viruses already known. The classification and nomenclature of the latest known viruses appear in reports of the International Committee on the Taxonomy of Viruses (ICTV), 7th edition (van Regenrmortel et al., editors. Seventh ICTV report. San Diego: Academic Press; 2000.) The basic viral hierarchical classification scheme is: Order, Family, Subfamily, Genus, Species, Strain, and Type as set out below.

Virus orders represent groupings of families of viruses that share common characteristics and are distinct from other orders and families. Virus orders are designated by names with the suffix-virales. Virus families are designated by names with the suffix-viridae. Virus families represent groupings of genera of viruses that share common characteristics and are distinct from the member viruses of other families. Viruses are placed in families on the basis of many features. A basic characteristic is nucleic acid type (DNA or RNA) and morphology, that is, the virion size, shape, and the presence or absence of an envelope. The host range and immunological properties (serotypes) of the virus are also used. Physical and physicochemical properties such as molecular mass, buoyant density, thermal inactivation, pH stability, and sensitivity to various solvents are used in classification. Virus genera represent groupings of species of viruses that share common characteristics and are distinct from the member viruses of other genera. Virus genera are designated by terms with the suffix-virus. A virus species is defined as a polythetic class of viruses that constitutes a replicating lineage and occupies a particular ecological niche.

Some viral families and their respective, sub-families, genera, and species contemplated for inactivation by contact and adsorption by the clays described herein include, but are not limited to, the following viruses set out in Tables 1-3 below.

TABLE 1
DNA VIRUSES

| Family | Sub-Family | Genus | Virus |
|---|---|---|---|
| Herpesviridae | Alphaherpesvirinae | Simplexvirus | Herpes simplex type |
| | | | 1 (HHV-1) |
| | | | Herpes simplex type |
| | | | 2 (HHV-2) |
| | | Varicellovirus | Varicella zoster virus |
| | | | (HHV-3) |
| | Betaherpesvirinae | Cytomegalovirus | Cytomegalovirus |
| | | | virus (HHV-5) |
| | | Roseolovirus | Human herpes virus |
| | | | type 6, 7 |
| | Gammaherpesvirinae | Lymphocryptovirus | Epstein Barr virus |
| | | | (HHV-4) |
| | | Rhadinovirus | Human herpes virus |
| | | | type 8 |

| | | |
|---|---|---|
| Poxviridae | Orthopoxvirus | Variola virus |
| | Molluscipoxvirus | Molluscum |
| | | contagiousum virus |
| Adenoviridae | Mastadenovirus | Human adenovirus |
| Papovaviridae | Papillomavirus | Papillomavirus |
| | Polyomavirus | BK virus |
| | | JC virus |
| Parvoviridae | Erythrovirus | Human parvovirus |
| | | (B 19) |

TABLE 2
RNA VIRUSES

| Family | Genus | Virus |
|---|---|---|
| Picornaviridae | Rhinovirus | Rhinovirus |
| | Hepatovirus | Hepatitis A virus |
| | Rubivirus | Rubella virus |
| | Alphavirus | Eastern equine encephalitis virus |
| | Rhadinovirus | Human herpes virus type 8 |
| Togaviridae | Flavivirus | Yellow fever virus |
| | | Dengue virus |
| | | West Nile virus |
| Flaviviridae | Hepacvirus | Hepatitis C virus |
| | Coronavirus | Human coronavirus |
| | Calicivirus | Norwalk virus |
| | Rubulavirus | Mumps virus |
| Coronaviridae | Morbillivirus | Measles virus |
| Caliciviridae | Pneumovirus | Respiratory syncitial virus (RSV) |
| Paramyxoviridae | Paramyxovirus | Human parainfluenza virus 1 |
| | Lyssavirus | Rabies virus |
| | Filovirus | Ebola virus |
| | Arenavirus | Lassa fever virus |
| Rhabdoviridae | Influenzavirus A | Influenza A |
| Filoviridae | Influenzavirus B | Influenza B |
| Arenaviridae | Influenzavirus C | Influenza C |
| Orthomyxoviridae | Hantavirus | Sin Nombre virus |
| Bunyaviridae | | |

TABLE 3
DNA-RNA REVERSE TRANSCRIBING VIRUSES

| Family | Genus | Virus |
|---|---|---|
| Retroviridae | Lentivirus | Human immunodeficiency viruses |
| | BLV-HTLV | Human T-cell leukemia viruses |
| | retroviruses | |
| Hepadnaviridae | Orthohepadnavirus | Hepatitis B virus |

Therapeutic Uses for the Layered Phyllosilicate Material

In yet another embodiment, the invention provides various in vitro and in vivo methods of using the layered phyllosilicates of the invention. Generally speaking, the layered phyllosilicates of the invention are useful for inactivating viruses as well as for providing a delivery vehicle for vaccines, therapeutics and/or diagnostic agents.

In one aspect, inactivation of a virus using the layered phyllosilicate material described herein is by one or more mechanisms selected from the group consisting of adsorption, ionic complexing, electrostatic complexing, chelation, hydrogen bonding, ion-dipole, dipole/dipole, Van Der Waals forces, and any combination thereof. Such ionic bonding provides inactivation of a virus molecule by a phyllosilicate material. Viral inactivation prevents a virus from migrating to and penetrating cell membranes, thereby preventing the virus from reproducing and rupturing the cells and releasing more of the virus to attach to and infect host cells. Accordingly, the layered phyllosilicate material inhibits virus entry and fusion to host cells and provides a physical barrier between a virus and a host cell.

The use of the layered phyllosilicate material described herein for the inactivation of both enveloped and non-enveloped viruses is contemplated. An enveloped virus comprises a capsid surrounded by a lipid bilayer derived from a membrane of the host cell and membrane proteins involved in adsorption found in the envelope. Non-enveloped viruses lack this lipid bilayer surrounding the capsid and have the proteins associated with adsorption found directly on (or part of) the capsid. Because the layered phyllosilicate material interacts directly with the oppositely charged surface of a virus, the presence of the lipid envelope on an enveloped virus is not expected to affect this interaction. The oppositely charged molecules on the surface of a virus include proteins, glycoproteins, lipids and combinations thereof. Further, because the layered phyllosilicate material interacts with the oppositely charged molecules on the surface of a virus, and not the genetic material in the nucleus of the virus, the inactivation of a virus by the layered phyllosilicate material is not affected by mutation, antigenic drift, or genetic recombination of the virus. Accordingly, a method of preventing a virus from becoming resistant to a particular material, comprising contacting a virus with a material that interacts with the oppositely charged molecules of the virus is specifically contemplated. In one aspect, the interaction between the layered phyllosilicate material and the oppositely charged molecules of the virus is a mechanism selected from the group consisting of adsorption, ionic complexing, electrostatic complexing, chelation, hydrogen bonding, ion-dipole, dipole/dipole, Van Der Waals forces and combinations thereof.

Thus, in one embodiment, the invention includes a method of inactivating a virus by contacting the virus with a layered phyllosilicate as described herein in an amount effective to inactivate the virus. The method can either be an in vitro method or an in vivo method. In one aspect, a composition comprising a layered phyllosilicate is administered to a mammal to inactivate a virus in waste expelled from said mammal. In some aspects, the waste is fecal matter. In other aspects, the waste is urine. Because the virus contained in the waste is inactive when expelled from the mammal, the virus is unable to infect mammals that come in contact with the expelled waste.

The invention also provides a method of treating a mammalian subject having a viral infection comprising administering an effective amount of a composition comprising a layered phyllosilicate material. In some embodiments, the mammalian subject is an animal. Exemplary animals include, but are not limited to, farm animals such as horses, cows, sheep, pigs, alpacas, llamas and goats; companion animals such as dogs and cats; exotic and/or zoo animals; laboratory animals including mice, rats, rabbits, guinea pigs and hamsters; poultry such as chickens, turkey, ducks and geese and other birds. In other embodiments, the mammalian subject is a human. Exemplary viral infections include, but are not limited to, those affecting the respiratory system (e.g., pharyngitis, tonsilitis, sinusitis and otitis media, influenza, laryngo-tracheo Bronchitis (croup), acute bronchitis, acute bronchiolitis, pneumonia and bronchopneumonia.), gastrointestinal tract, brain and spinal cord (central nervous system) and the skin.

In addition, the layered phyllosilicate material can be used to treat a variety of other conditions. In one embodiment, the condition is a skin condition including, but not limited to, a bacterial skin condition, a microbial skin condition, a biofilm skin condition, an inflammatory skin condition, a hyperproliferative skin condition, a fungal skin condition, a viral skin condition, an autoimmune skin condition, an idiopathic skin condition, a hyperproliferative skin condition, a cancerous skin condition. Exemplary skin conditions include, but are not limited to a burn, eczema (including, but not limited to, atopic eczema, acrodermatitis continua, contact allergic dermatitis, contact irritant dermatitis, dyshidrotic eczema, pompholyx, lichen simplex chronicus, nummular eczema, seborrheic dermatitis, stasis eczema), erythroderma, an insect bite, mycosis fungoides, pyoderma gangrenosum, eythrema multiforme, rosacea, onychomycosis, acne (including, but not limited to, acne vulgaris, neonatal acne, infantile acne, pomade acne), psoriasis, Reiter's syndrome, pityriasis rubra pilaris, hyperpigmentation, vitiligo, scarring conditions, keloid, lichen planus, age-related skin disorder (including, but not limited to, wrinkles and cellulite) and hyperproliferative skin disorders, including, but not limited to, hyperproliferative variants of the disorders of keratinization (including, but not limited to, actinic keratosis, senile keratosis). As an example, the metal-containing material can be used prophylactically to reduce (e.g., prevent) the occurrence of a particular burn (e.g., a second degree burn) becoming a more severe burn (e.g., a third degree burn).

In another embodiment, the condition is a respiratory condition including, but not limited to a bacterial respiratory condition, a biofilm respiratory condition, a microbial respiratory condition, an inflammatory respiratory condition, a fungal respiratory condition, a viral respiratory condition, an autoimmune respiratory condition, an idiopathic respiratory condition, a hyperproliferative respiratory condition, a cancerous respiratory condition. Exemplary respiratory conditions include, but are not limited to, asthma, emphysema, bronchitis, pulmonary edema, acute respiratory distress syndrome, bronchopulmonary dysplasia, fibrotic conditions, pulmonary fibrosis, pulmonary atelectasis, tuberculosis, pneumonia, sinusitis, allergic rhinitis, pharyngitis, mucositis, stomatitis, chronic obstructive pulmonary disease, bronchiectasis, lupus pneumonitis and cystic fibrosis.

In another embodiment, the condition is a musculo-skeletal condition, including but not limited to, a bacterial musculo-skeletal condition, a biofilm musculo-skeletal condition, a microbial musculo-skeletal condition, an inflammatory musculo-skeletal condition, a fungal musculo-skeletal condition, a viral musculo-skeletal condition, an autoimmune musculo-skeletal condition, an idiopathic musculo-skeletal condition, a hyperproliferative musculo-skeletal condition, a cancerous musculo-skeletal condition. The musculo-skeletal condition can be, for example, a degenerative musculo-skeletal condition (including arthritis) or a traumatic musculo-skeletal condition (including a torn or damaged muscle). Exemplary musculo-skeletal conditions include, but are not limited to tendonitis, osteomyelitis, fibromyalgia, bursitis and arthritis.

In another embodiment, the condition is a circulatory condition including, but not limited to a bacterial circulatory condition, a biofilm circulatory condition, a microbial circulatory condition, an inflammatory circulatory condition, a fungal circulatory condition, a viral circulatory condition, an autoimmune circulatory condition, an idiopathic circulatory condition, a hyperproliferative circulatory condition, a cancerous circulatory condition. As referred to herein, circulatory conditions include lymphatic conditions. Examples of circulatory conditions include arteriosclerosis, lymphoma, septicemia, leukemia, ischemic vascular disease, lymphangitis and atherosclerosis.

In yet another embodiment, the condition is a mucosal or serosal condition including, but not limited to a bacterial mucosal or serosal condition, a biofilm mucosal or serosal condition, a microbial mucosal or serosal condition, an inflammatory mucosal or serosal condition, a fungal mucosal or serosal condition, a viral mucosal or serosal condition, an autoimmune mucosal or serosal condition, an idiopathic mucosal or serosal condition, a hyperproliferative mucosal or serosal condition, a cancerous mucosal or serosal condition. Exemplary mucosal or serosal conditions include, but are not limited to pericarditis, Bowen's disease, stomatitis, prostatitis, sinusitis, allergic rhinitis, digestive disorders, peptic ulcers, esophageal ulcers, gastric ulcers, duodenal ulcers, espohagitis, gastritis, enteritis, enterogastric intestinal hemorrhage, toxic epidermal necrolysis syndrome, Stevens Johnson syndrome, cystic fibrosis, bronchitis, pneumonia, pharyngitis, common cold, ear infections, sore throat, sexually transmitted diseases (including, but not limited to syphilis, gonorrhea, herpes, genital warts, HIV, chlamydia), inflammatory bowel disease, colitis, hemorrhoids, thrush, dental conditions, oral conditions, conjunctivitis, and periodontal conditions.

In one embodiment, a composition comprising a layered phyllosilicate material will further comprise a therapeutic agent. The therapeutic agent may be a small molecule or macromolecule such as peptide, protein or nucleic acid. The therapeutic agents may be selected from the group consisting of carrageenan, anti-inflammatory agents, including hydrocortisone, prednisone, and the like; NSAIDS, including acetaminophen, salicylic acid, ibuprofen, and the like; selective COX-2 enzyme inhibitors, antibacterial agents, including colloidal silver, penicillin, erythromycin, polymyxin B, viomycin, chloromycetin, streptomycins, cefazolin, ampicillin, azactam, tobramycin, cephalosporins, bacitracin, tetracycline, doxycycline, gentamycin, quinolines, neomycin, clindamycin, kanamycin, metronidazole, and the like; antiparasitic agents including quinacrine, chloroquine, vidarabine, and the like; antifungal agents including nystatin, and the like; anti-virucides, and antiviral agents including acyclovir, docosanol, ribarivin, interferons, and the like; cellulose acetate, carbopol and carrageenan (CAS No. 9000-07-1); systemic analgesic agents including salicylic acid, acetaminophen, ibuprofen, naproxen, piroxicam, flurbiprofen, morphine, and the like; local anesthetics including cocaine, lidocaine, bupivacaine, xylocalne, benzocaine, and the like; an antisense nucleotide, a thrombin inhibitor, an antithrombogenic agent, a tissue plasminogen activator, a thrombolytic agent, a fibrinolytic agent, a vasospasm, inhibitor, a calcium channel blocker, a nitrate, a nitric oxide promoter, a vasodilator, an antimicrobial agent, an antibiotic, an anti-platelet agent, an anti-mitotic, a microtubule inhibitor, an actin inhibitor, a remodeling inhibitor, an agent for molecular genetic intervention, a cell cycle inhibitor, an inhibitor of the surface glycoprotein receptor, an anti-metabolite, an anti-proliferative agent, an anti-cancer chemotherapeutic agent, an anti-inflammatory steroid, an immunosuppressive agent, an antibiotic, a radiotherapeutic agent, iodine-containing compounds, barium-containing compounds, a heavy metal functioning as a radiopaque agent, a peptide, a protein, an enzyme, an extracellular matrix component, a cellular component, a biologic agent, an angiotensin converting enzyme (ACE) inhibitor, ascorbic acid, a free radical scavenger, an iron chelator, an antioxidant, a radiolabelled form or other radiolabelled form of any of the foregoing, or a mixture of any of these.

In another embodiment, a layered phyllosilicate material is used prophylactically as a vaccine. Without being bound to any particular mechanism, virus inactivation by layered phyllosilicates occurs upon binding of the layered phyllosilicate to certain glycoprotein receptors on the virus surface, thereby preventing the virus from attaching to receptors on T cells in the body. Accordingly, the layered phyllosilicates, with the inactivated virus adsorbed thereto, is administered to a subject in need of prophylactic treatment. In another embodiment, antigenic epitopes and other immunogenic compounds can be incorporated within the layers of the phyllosilicates and administered to a mammalian subject in need of prophylactic treatment. See, for example, U.S. Pat. No. 6,475,595 and U.S. Patent Application Publication No. 2006/0177468.

In yet another embodiment, a layered phyllosilicate material is utilized as delivery vehicle. In one embodiment, the layered phyllosilicates are a delivery vehicle for nucleic acids and proteins. For example, binding of proteins to negatively-charged layered phyllosilicates can neutralize the charge and/or induce conformational changes of the proteins and thus promote their permeability and absorption through mucosal membranes. Similarly, nucleic acids (e.g., DNA, RNA, RNAi and antisense oligonucleotides) intercalated within positively-charged layered phyllosilicates can enter desired cells via phagocytosis and endocytosis and will also promote mucosal membrane absorption and permeability. In one embodiment, the layered phyllosilicates are the delivery vehicle. In other embodiments, the layered phyllosilicates are used in lieu of or in conjunction with other nucleic acid and protein delivery vehicles known in the art in order to increase the delivery potential and cell targeting or membrane permeability and absorption of these molecules. Exemplary nucleic acid and protein delivery vehicles known in the art include, but are not limited to, U.S. Pat. Nos. 7,098,032; 7,029,697; 6,962,686; 6,919,091; 6,916,490; 6,897,068; 6,890,556; 6,821,955; 6,764,853; 6,740,643; 6,475,995; 6,468,986; 6,458,382; 6,426,086; 6,409,990; 6,379,966; 6,383,478; 6,365,575; 6,344,436; 6,319,715; 6,287,857; 6,217,912; 6,207,456; 6,184,037; 6,077,835; 6,008,336; 5,972,900; 5,972,707; 5,948,878; 5,877,302; 5,844,107; 5,820,879; 5,759,519; 5,283,185; and U.S. Patent Application Publication Nos. 2006/0105371 2006/0084617, 2006/0051405, 2005/0265956, 2005/0260276, 2005/0234232, 2005/0123600, 2005/0090008, 2005/0027064, 2005/0008617, 2004/0156909, 2003/0199090, 2003/0026841, the disclosures of which are incorporated herein by reference in their entireties.

In another embodiment, binding of small molecule drugs to a layered phyllosilicate material can improve their delivery and absorption through mucosal membranes, including the ocular, dermal, nasal and intestinal membranes. Drug release from the layered phyllosilicates can be induced by pH, ionic strength changes, and/or in response to temperature, ionic current or ultrasound. In one embodiment, the layered phyllosilicates are the delivery vehicle. In other embodiments, the layered phyllosilicates are used in lieu of or in conjunction with other small molecule drug delivery vehicles known in the art in order to increase cell targeting membrane permeability and absorption. Exemplary small molecule drug delivery systems known in the art include, but are not limited to, those described in U.S. Pat. Nos. 6,838,528; 6,797,704; 6,730,334; 6,706,289; 6,482,439; 6,443,989; 6,383,478; 6,165,440; 5,780,044; 5,759,563; 5,565,215; and U.S. Patent Application Publication Nos. 2006/0193787; 2006/0149392; 2006/0105049; 2006/0057206; 2006/0034925; 2005/0266090; 2005/0260276; 2005/0249774; 2005/0220754; 2005/0058603, the disclosures of which are incorporated herein by reference in their entireties.

In other embodiments of the invention, a layered phyllosilicate material is used to coat, or is impregnated within, a device including a medical stents and the like. For devices that are coated, the coating process is performed in such a manner as to (a) coat only one surface of device with the compositions of the invention or (b) coating all or parts of the device with the compositions of the invention.

The layered phyllosilicate material-based coating or device coated with the same is made sterile either by preparing the layered phyllosilicate material-based coating or device coated with the same under aseptic environment and/or may be terminally sterilized using methods known in the art, such as ethanol, ethylene oxide, gamma radiation or electron beam sterilization methods or a combination of both of these methods.

Thus, a therapeutic agent is advantageously delivered to adjacent tissues or tissues proximal to the implant site. In one aspect, the therapeutic agent is the layered phyllosilicate material. The layered phyllosilicates may be used alone or in combination with another therapeutic agent described herein. The therapeutic agent is capable of being released from the solid implanted matrix into adjacent or surrounding tissue fluids during biodegradation, bioerosion, or bioresorption of the fixation device.

Other agents also may be used in the coating compositions of the invention. Preferably, such agents are capable of preventing infection in the host, either systemically or locally at the defect site, are contemplated as illustrative useful additives. Exemplary additives include the therapeutic agents described herein.

In yet another aspect, the implantable device coatings made from the compositions of the invention may be used for delivering a specific therapeutic or other agent to an external portion (surface) of a body passageway or cavity. Examples of body passageways include arteries, veins, the heart, the esophagus, the stomach, the duodenum, the small intestine, the large intestine, biliary tracts, the ureter, the bladder, the urethra, lacrimal ducts, the trachea, bronchi, bronchiole, nasal airways, Eustachian tubes, the external auditory mayal, vas deferens and fallopian tubes. Examples of cavities include the abdominal cavity, the buccal cavity, the peritoneal cavity, the pericardial cavity, the pelvic cavity, perivisceral cavity, pleural cavity and uterine cavity.

Controlled drug delivery matrices can be in the form of a patch, implantable or insertable medical device incorporating drug intercalated layered phyllosilicates. Specific organic macromolecules such as surfactants and polymers can be used to provide the desired drug release rate. Suitable surfactants and polymers are well known to those skilled in the art. See, for example, U.S. Patent Application Publication No. 2005/0208122, the disclosure of which is incorporated herein by reference in its entirety.

Other Medical Uses for the Layered Phyllosilicates

In yet another embodiment, a layered phyllosilicate material is used to screen for drug candidates. For example, drug candidates can be adsorbed onto layered phyllosilicates and then used in receptor-binding studies to identify lead candidate molecules for drug development. Alternatively, drug receptors can be incorporated into layered phyllosilicates and then employed in drug-receptor binding studies, provided however, that adsorption of receptors onto bentonite clay does not alter their drug binding affinity.

In another embodiment, a layered phyllosilicate material is used as a substrate to adsorb certain membrane proteins and receptors from cell surfaces as part of a purification process (e.g., affinity chromatography). Affinity chromatography is used to separate proteins by selective adsorption onto and/or elution from a solid medium, generally in the form of a column. The solid medium is usually an inert carrier matrix to which is attached a ligand having the capacity to bind, under certain conditions, the target or desired protein or proteins over others present in the same sample, although in some cases the matrix itself may have such selective binding capacity. The ligand may be biologically complementary to the protein to be separated, for example, antigen and antibody, or may be any biologically unrelated molecule which, by virtue of the nature and steric relationship of its active groups, has the ability to bind the protein. Examples of commonly used affinity chromatography techniques include immobilized metal affinity chromatography (IMAC), sulfated affinity chromatography, dye affinity chromatography, and heparin affinity. In another example, the chromatographic medium may be prepared using one member of a binding pair, e.g., a receptor/ligand binding pair, or antibody/antigen binding pair (immunoaffinity chromatography).

In some embodiments, a layered phyllosilicate material is used for medical imaging by associating an imaging component with the layered phyllosilicate. The present invention is not limited by the nature of the imaging component used. In some embodiments, the imaging is based on the passive or active observation of local differences in density of selected physical properties of the investigated complex matter. These differences may be due to a different shape (e.g., mass density detected by atomic force microscopy), altered composition (e.g., radiopaques detected by X-ray), distinct light emission (e.g., fluorochromes detected by spectrophotometry), different diffraction (e.g., electron-beam detected by transmission electron microscopy), contrasted absorption (e.g., light detected by optical methods), or special radiation emission (e.g., isotope methods). Thus, quality and sensitivity of imaging depend on the property observed and on the technique used.

In one embodiment, imaging agents can be incorporated into layered phyllosilicates targeted to tumors to detect cancer.

In yet another embodiment, a layered phyllosilicate material is used as a diagnostic agent to detect and quantify certain analytes present in biological specimens including blood, plasma, saliva and urine. These analytes include small molecules or macromolecules such as proteins and enzymes, the levels of which are altered in disease states.

Routes of Administration and Dosage

The phyllosilicate compositions alone or in combination as described herein are administered by any route that delivers an effective dosage to the desired site of action, with acceptable (preferably minimal) side-effects. Numerous routes of administration are known, including for example, oral, rectal, vaginal, transmucosal, buccal or intestinal administration; parenteral delivery, including intraperitoneal, intramuscular, subcutaneous, intramedullary injections, as well as intrathecal, cutaneous or intradermal injections; respiratory or inhalation, nasal, pulmonary and topical application, including ocular and transdermal applications.

When used in the above or other treatments, a “therapeutically effective amount” or an “effective amount” of a layered phyllosilicate material or a composition comprising a layered phyllosilicate material means a sufficient amount of the layered phyllosilicate material is provided to treat disorders, at a reasonable benefit/risk ratio applicable to any medical treatment. It will be understood, however, that the total daily usage of the layered phyllosilicate material will be decided by the attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of a layered phyllosilicate material administered to a mammalian subject range from about 0.001 to about 200 mg/kg/day. If desired, the effective daily dose may be divided into multiple doses for purposes of administration; consequently, single dose compositions may contain such amounts or submultiples thereof to make up the daily dose. The dosage regimen of a phyllosilicate composition alone or in combination as described herein to be used in antiviral treatment will be determined by the attending physician considering various factors which modify the action of the phyllosilicate, e.g., the patient's age, sex, and diet, the severity of any infection, time of administration and other clinical factors.

Oral dosage forms include tablets, capsules, caplets, solutions, suspensions and/or syrups, and may also comprise a plurality of granules, beads, powders or pellets that may or may not be encapsulated. Such dosage forms are prepared using conventional methods known to those in the field of pharmaceutical formulation and described in the pertinent texts, e.g., in Remington: The Science and Practice of Pharmacy, supra). Tablets and capsules represent the most convenient oral dosage forms, in which case solid pharmaceutical carriers are employed.

Tablets may be manufactured using standard tablet processing procedures and equipment. One method for forming tablets is by direct compression of a powdered, crystalline or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material.

In addition to the layered phyllosilicate material alone or in combination as described herein, tablets prepared for oral administration will generally contain other materials such as binders, diluents, lubricants, disintegrants, fillers, stabilizers, surfactants, preservatives, coloring agents, flavoring agents and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum. Diluents are typically necessary to increase bulk so that a practical size tablet is ultimately provided. Suitable diluents include dicalcium phosphate, calcium sulfate, lactose, cellulose, kaolin, mannitol, sodium chloride, dry starch and powdered sugar. Lubricants are used to facilitate tablet manufacture; examples of suitable lubricants include, for example, vegetable oils such as peanut oil, cottonseed oil, sesame oil, olive oil, corn oil, and oil of theobroma, glycerin, magnesium stearate, calcium stearate, and stearic acid. Disintegrants are used to facilitate disintegration of the tablet, and are generally starches, clays, celluloses, algins, gums or crosslinked polymers. Fillers include, for example, materials such as silicon dioxide, titanium dioxide, alumina, talc, kaolin, powdered cellulose and microcrystalline cellulose, as well as soluble materials such as mannitol, urea, sucrose, lactose, dextrose, sodium chloride and sorbitol. Stabilizers are used to inhibit or retard drug decomposition reactions that include, by way of example, oxidative reactions. Surfactants may be anionic, cationic, amphoteric or nonionic surface active agents.

The dosage form may also be a capsule, in which case the layered phyllosilicate material-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. (See, for e.g., Remington: The Science and Practice of Pharmacy, supra), which describes materials and methods for preparing encapsulated pharmaceuticals.

Solid dosage forms, whether tablets, capsules, caplets, or particulates, may, if desired, be coated so as to provide for delayed release. Dosage forms with delayed release coatings may be manufactured using standard coating procedures and equipment. Such procedures are known to those skilled in the art and described in the pertinent texts (See, for e.g., Remington: The Science and Practice of Pharmacy, supra). Generally, after preparation of the solid dosage form, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained release dosage forms provide for drug release over an extended time period, and may or may not be delayed release. Generally, as will be appreciated by those of ordinary skill in the art, sustained release dosage forms are formulated by dispersing a drug within a matrix of a gradually bioerodible (hydrolyzable) material such as an insoluble plastic, a hydrophilic polymer, or a fatty compound, or by coating a solid, drug-containing dosage form with such a material. Insoluble plastic matrices may be comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

Although the present compositions may be administered orally, other modes of administration are contemplated as well. Exemplary modes of administration include transmucosal (e.g., U.S. Pat. Nos. 5,288,498; 6,248,760; 6,355,248; 6,548,490, the disclosures of which are incorporated herein by reference in their entireties), transurethral (e.g., e.g., U.S. Pat. Nos. 5,919,474 and 5,925,629, the disclosures of which are incorporated herein by reference in their entireties), vaginal or perivaginal (e.g., U.S. Pat. Nos. 4,211,679; 5,491,171 and 6,576,250, the disclosures of which are incorporated herein by reference in their entireties) and intranasal or inhalation (e.g., U.S. Pat. Nos. 4,800,878; 5,112,804; 5,179,079; 6,017,963; 6,391,318 and 6,815,424, the disclosures of which are incorporated herein by reference in their entireties). One of skill in the art would be able to modify a composition comprising a layered phyllosilicate material alone or in combination as described herein to be used in any of the modes of administration described herein.

The compositions comprising a layered phyllosilicate alone or in combination as described herein can also be used as a topical agent. Preferably, the topical agent is a solution, that is, a liquid formulation comprising the layered phyllosilicate material and a carrier. Other suitable forms include semi-solid or solid forms comprising a carrier indigenous to topical application and having a dynamic viscosity preferably greater than that of water, provided that the carrier does not deleteriously react with the layered phyllosilicate material in the composition. Suitable formulations include, but are not limited to, lip balms, suspensions, emulsions, creams, ointments, powders, liniments, salves and the like. If desired, these may be sterilized or mixed with auxiliary agents, e.g., preservatives, stabilizers, wetting agents, buffers or salts for influencing osmotic pressure and the like. Preferred vehicles for semi-solid or solid forms topical preparations include ointment bases, e.g., polyethylene glycol-1000 (PEG-1000); conventional ophthalmic vehicles; creams, (e.g., HEB cream); and gels, (e.g., K-Y gel); as well as petroleum jelly and the like. These topical preparations may also contain emollients, perfumes, and/or pigments to enhance their acceptability for various usages, provided that the additives do not deleteriously react with the layered phyllosilicate material in the composition.

Also suitable for topical application are sprayable aerosol preparations wherein the layered phyllosilicate material, preferably in combination with a solid or liquid inert carrier material, is packaged in a squeeze bottle or in admixture with a pressurized volatile, normally gaseous propellant, e.g., a Freon (chlorofluorocarbon) or environmentally acceptable volatile propellant. Such compositions can be used for application to environmental surfaces, e.g., examining tables, toilet seats and the like, and/or for application to the skin or to mucous membranes. The aerosol or spray preparations can contain solvents, buffers, surfactants, perfumes, and/or antioxidants in addition to the layered phyllosilicate material of the invention.

The compositions of this invention can be employed in mixture with conventional excipients, i.e., pharmaceutically acceptable organic or inorganic carrier substances suitable for topical application which do not deleteriously react with the acid or the alcohol in the composition. The compositions of the invention can also include diluents, fillers, salts, buffers, stabilizers, solubilizers, and other materials well known in the art (Remington's Pharmaceutical Sciences 16th edition, Osol, A. Ed. (1980).

The composition also may be formulated as a dispersable powder for dusting the skin, hair, fur, or feathers of humans or animals. The compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents and scent enhancers.

The layered phyllosilicate material of the invention can be administered in a concentration (w/v) ranging from about 0.1% to about 20%, or from about 1% to about 10%, or in a concentration of about 2%, about 3%, about 4%, about 5%, about 6%, about 7%, about 8%, about 9%, about 10%, about 11%, about 12%, about 13%, about 14%, about 15%, about 16%, about 17%, about 18%, about 19% or about 20%.

Kits and Unit Doses

In related variations of the preceding embodiments, a composition comprising a layered phyllosilicate material alone or in combination as described herein may be so arranged, e.g., in a kit or package or unit dose, to permit co-administration with one or more other therapeutic agents, but the layered phyllosilicate material composition and the agent are not in admixture. In another aspect, the layered phyllosilicate material composition and the agent are in admixture. In some embodiments, the two components to the kit/unit dose are packaged with instructions for administering the two agents to a human subject for treatment of one of the above-indicated disorders and diseases. The kit may comprise the composition of the invention in combination with a vehicle in a cream or gel base, as a pump-spray, as an aerosol, on an impregnated bandage, a medicated animal ear tag or collar, or in a dropper. The composition of the invention may also be in any one of the above formulations in combination with a second agent, including but not limited to antiviral agents, topical steroids, aloe vera and the like cosmeceuticals. In one aspect, the kit includes applicator for administering the composition.

With respect to diagnostic applications of layered phyllosilicates, suitable diagnostic kits and reagents known in the art can be employed. This may include incorporation of the layered phyllosilicates into a diagnostic dipstick or a device followed by certain color, conductivity or electric current changes upon contact with a biological specimen.

Veterinary Applications

Materials and methods of the invention can be practiced on animals of economic value, to treat animal viral infections and other skin conditions. Treatment of any domestic pet animal, livestock, zoo animals, circus animals, endangered species, and the like is specifically contemplated.

Poxyiridae virus infection occurs in many animal species important as livestock or pets, causing disease in these animals similar to human disease, which at times can result in serious side effects to the animal or livestock industry. For example, the Cowpox virus which is harbored originally in rodents, can spread to cats, cows, humans, and zoo animals, including large cats and elephants. Transmission to humans traditionally occurs via contact with the infected teats of milking cows. However, infections are currently seen more commonly among domestic cats, from which cowpox can be transmitted to humans. Cowpox infection is a self-limiting disease resulting in vesicles and pustules of the hands in humans and similar areas in animals.

Pseudocowpox virus, the agent of pseudocowpox (Milker's nodules, paravaccinia), causes an epithelial cell infection in handlers of cows. Orf virus infection results in painful lesions on the skin of sheep, and goats, and can be serious for lambs whose mouth lesions stop them from feeding. Sheep pox and goat pox may be fatal infections, with visceral as well as dermal lesions. Seal pox may result in a severe skin and flipper infection of captive and wild seals. Myxomatosis infects rabbits, and is typically fatal to the infected animal. Yaba monkey tumor virus causes a histiocytoma, or subcutaneous tumorlike growths, of the head or limbs of primates, especially African monkeys, which are often seen in zoos and are important in biological studies. Tanapox virus causes tanapox, a self-limiting epithelial cell infection in primates. Other viruses include pig pox, cat pox, camel pox, Fowl pox, pigeon pox, canary pox, and Ectromelia, which infects mice.

Other aspects and advantages of the present invention will be understood upon consideration of the following illustrative examples, which are not intended to be limiting in any way.

### Examples

### Example 4

### Antiviral Activity of Test Compounds Against HIV-1

In this study, three different compositions of bentonite clay were studied (R-0088, R-0089, and R-0090) to evaluate their adsorption and antiviral efficacy against an HIV-1 virus (Retroscreen Virology Ltd). Each bentonite clay composition was studied at three different concentrations (0.01% w/v, 0.001% w/v, and 0.0001% w/v) prepared in sterile double-distilled water) and at three different incubation times (1 minute, 5 minutes, and 10 minutes). Test compositions composed of various mineral clays and controls (as listed below) were prepared.
R-0088—purified homoionic sodium bentonite mixture, purified in accordance with U.S. Pat. No. 6,050,509.
R-0089—purified acid activated clay mixture.
R-0091—purified bentonite:dextran analog modified mixture.
C8166 growth media (negative control)
20% Ethanol/PBS (positive control)

HIV-1IIIB (AL307 with a titer of 104TCID5O/ml) was supplied from the Retroscreen Virology Ltd virus repository. Virucidal and P24 assays were carried out as set out below to evaluate antiviral activity. The p24 antigen assay measures the viral capsid (core) p24 protein in blood that is detectable earlier than HIV antibody during acute infection.

Virucidal Assay

1. 40 μl of the viral stock solution was added to each concentration of test compound (360 μl) and left to incubate at room temperature for the incubation times specified above.
2. The reaction was terminated by the addition of cell infection media (3.6 ml), which diluted the reaction 10-fold.
   P24 Assay
1. The samples were left to settle for 1.5 hours before being added to the P24 antigen coated plates.
2. 200 μl of each sample was added to the assay plate.
3. 110 μl of neat stock virus (AL307) was added to the relevant wells on the plate.
4. Empigen (final concentration of 0.8%) was added to all these wells.
5. The neat stock virus was titrated across the wells following a 10-fold dilution series in RPMI-1640 containing 1% Empigen.
6. The P24 assay was then conducted as instructed in the current Retroscreen Virology Ltd. SOP.

Only R-0088 at 0.01% w/v concentration reduced the viral titer of HIV-1_{IIIB} at the minute incubation time with 99.13% efficacy exhibited. Virucidal results for R-0088 demonstrated that a time-response is exhibited by the 0.01% w/v concentration. At this concentration, the reduction in the HIV1_{IIIB} virus titer was significant at the 10 minute incubation time with a reduction of 2.29 logs. A reduction of ≧1-log₁₀ TCID₅₀/ml (Oxford et al, Antiv. Chem. Chemother. 5:176-181, 1994) is deemed significant for the virucidal assays used in this study, and is equivalent to ≧90% reduction in viral titer. Virucidal results for R-0089 and R-0091 did not demonstrate significant reductions in HIV-1_{IIIB} titer.

At the highest test concentration (0.01% w/v), R-0088 exhibited a significant reduction in the HIV-1_{IIIB} (AL307 with a titer of 10⁴TCID₅₀/ml). R-0089 and R-0091 did not exhibit significant reductions in the HIV1_{IIIB} virus titer for any of the variables tested.

### Example 5

### Antiviral Activity of Test Compounds Against Influenza A

This Study was performed to determine whether the test compounds have virucidal efficacy against an epidemic strain of Influenza A virus and to assess the cytotoxic potential of the test compounds on Madin-Darby canine kidney cells (MDCK) cells. Three different compositions of bentonite clay (R-0088, R-0089, and R-0090) were studied to evaluate their adsorption and antiviral efficacy against an Influenza A/Panama/2007/99 (H₃N₂) virus.

Test compositions composed of various mineral clays and controls (as listed below) were prepared.
R-0088—purified sodium bentonite mixture, purified in accordance with U.S. 6,050,509.
R-0089—purified acid activated clay mixture.
R-0090—purified bentonite-sialic acid mixture.
C8166 growth media (negative control)
20% Ethanol/PBS (positive control)

Each bentonite clay mixture was studied at three different concentrations (0.01% w/v, 0.001% w/v, and 0.0001% w/v prepared in sterile double-distilled water) and at five different incubation times (30 seconds, 1 minute, 5 minutes, 10 minutes, and 30 minutes).

The cells of the toxicity controls were incubated with cell maintenance media, whereas the cells of the virucidal controls were incubated with cell infection media. The stock titer of Influenza A/Panama/2007/99 virus was 7.7 log₁₀ TCID₅₀/ml. Before use in the virucidal assay, the stock virus was diluted 100-fold in infection media. It was then diluted a further 2-fold when it was added to the reaction mixture (section 9.3.2, step 4). The resulting test titer as therefore 5.4 log₁₀ TCID₅₀/ml. The protocols for the toxicity assay and the virucidal assay are set out below.

Toxicity Assay

1. Cells (100 μl/well) at 1×10⁵ cells/ml were seeded onto 96-well plates and incubated at 37° C. for ˜24 hours.
2. The cell maintenance media on the plates was removed and the cell monolayer washed twice with PBS (100 μl/well).
3. Each test compound (100 μl/well) was added, in quadruplicate, to the plate and left to incubate at room temperature for the various times specified.
4. The test compounds were removed and the cell monolayer washed twice with phosphate buffered saline (PBS) (100 μl/well).
5. Cell maintenance media (100 μl/well) was added to the cell monolayer and the plates incubated at 37° C. for ˜24 hours
6. A crystal violet assay was performed on the plates in accordance to the Retroscreen Virology Ltd. SOP VA024-01.

Controls utilized in the toxicity assay were:
Cell only control: untreated cells. This was a negative control for toxic cytopathic effect (tCPE) and was also an indicator of cell quality.
Diluent control: cells treated with sterile double-distilled water for the specified times. This was a negative control for the test compounds and assessed any toxic effects of the diluent.
Cell and PBS control: untreated cells washed four times with PBS and incubated with cell maintenance media. This was a negative control for the washing steps, which involved a total of four washes with PBS.
   Virucidal Assay
1. Cells (100 μl/well) at 1×10⁵ cells/ml were seeded onto 96-well plates and incubated at 37° C. for ˜24 hours.
2. The cell maintenance media on the plates was removed and the cell monolayer washed twice with PBS (100 μl/well).
3. Cell infection media (100 μl/well) was added to the plates.
4. Diluted virus (200 μl) of 1/2000 viral stock solution was added to each test compound (200 μl) and left to incubate at room temperature for the various incubation times specified.
5. The reaction was terminated by the addition of cell infection media (3.6 ml), which diluted the reaction 10-fold.
6. The termination mixture was centrifuged (4000 rpm for 10 minutes) and the supernatant harvested.
7. The cell infection media in wells B4-B11 of the 96-well plate was removed. The supernatant (111 μl/well) was added to wells B8-B11, and the cell only control was added to wells B4-B7. Both were plated in quadruplicate.
8. The plates were incubated at 37° C. and 5% CO₂ for 2 days.
9. On day 2 post-infection, the plates were scored for viral cytopathic effect (vCPE) and a hemagglutination (HA) assay was performed as per Retroscreen Virology Ltd. SOP VA018-02.

Controls utilized in the virucidal assay were:
Cell only control: cells not infected with virus. This is a negative control for vCPE and is also an indicator of cell quality.
Virus only control: cells infected with a 1/2000 dilution of the virus stock. This was a positive control for vCPE.
Diluent control: cells infected with virus that was pre-treated with sterile double-distilled water for the specified times. This was a negative control for the test compounds and assessed any antiviral effects of the diluent.
Spun virus control: cells infected with virus that was centrifuged at 4000 rpm for 10 minutes. This was a negative control for the centrifugation step and assessed whether centrifugation affected viral titer.
Antiviral control: cells infected with virus pre-treated with citrate buffer at pH3.5. This was a positive control for the test compounds.

For the virucidal assay only, the test compounds were prepared at double the concentrations than those described above. This is due to the 2-fold dilution they underwent when they were mixed with the virus.

The virucidal results demonstrate that a time-response was exhibited by R-0088 at the 0.01% w/v concentration only. At this concentration, the reductions in the Influenza A/Panama/2007/99 virus titer by R-0088 were only significant for the 10 and 30 minute incubation times. R-0089 and R-0090 did not demonstrate significant reductions in the Influenza A/Panama/2007/99 virus titer.

Thus, at the highest test concentration (0.01% w/v), R-0088 exhibited a significant reduction in the Influenza A/Panama/2007/99 virus titer at the 10 and 30 minute incubation times. R-0089 and R-0090 did not exhibit significant reductions in the Influenza A/Panama/2007/99 virus titer for any of the variables tested.

### Example 6

### Antiviral Activity of Additional Test Compounds Against Influenza A

This study was performed to determine whether additional test compounds have virucidal efficacy against an epidemic strain of Influenza A virus and to assess the cytotoxic potential of these test compounds on Madin-Darby canine kidney cells (MDCK) cells. Three different compositions of bentonite clay were studied (R-100, R-101, and R-102) to evaluate their adsorption and antiviral efficacy against an Influenza A/Panama/2007/99 (H3N2) virus.

Test compositions composed of various mineral clays (as listed below) were prepared.
R-100—Crude sodium bentonite clay.
R-101—Sodium bentonite clay having non-smectite impurities removed (as in U.S. Pat. No. 6,050,509, but without the ion exchange steps).
R-102—Purified sodium bentonite clay, purified in accordance with U.S. Pat. No. 6,050,509.
C8166 growth media (negative control)
20% Ethanol/PBS (positive control)

Each bentonite clay mixture was studied at three different concentrations (0.01% w/v, 0.001% w/v, and 0.0001% w/v prepared in sterile double-distilled water) and at three different incubation times (10 minutes, 30 minutes, and 60 minutes).

The cells of the toxicity controls were incubated with cell maintenance media, whereas the cells of the virucidal controls were incubated with cell infection media. The stock titer of Influenza A/Panama/2007/99 virus was 7.4 log₁₀ TCID₅₀/ml. Before use in the virucidal assay, the stock virus was diluted 2000-fold in infection media. It was then diluted a further 2-fold when it was mixed with the test compounds, a further 10-fold when it was mixed with the anti-viral control. The protocols for the toxicity assay and the virucidal assay are set out below.

Toxicity Assay

The toxicity assay was performed as set out in Example 2 except for one modification; in step (1) of the assay, cells were seeded at (100 μl/well) at 5×104 cells/ml.

Controls utilized in the toxicity assay were:
Cell only control: untreated cells. This was a negative control for toxic cytopathic effect (tCPE) and was also an indicator of cell quality.
Diluent control: cells treated with sterile double-distilled water for the specified times. This was a negative control for the test compounds and assessed any toxic effects of the diluent.
PBS wash control: untreated cells washed four times with PBS and incubated with cell maintenance media. This was a negative control for the washing steps, which involved a total of four washes with PBS.
   Virucidal Assay
1. Cells (100 μl/well) at 5×10⁴ cells/ml or 7×10⁴ cells/ml were seeded onto 96-well plates and incubated at 37° C. for ˜24 hours.
2. The cell maintenance media on the plates was removed and the cell monolayer washed twice with PBS (100 μl/well).
3. Cell infection media (100 μl/well) was added to the plates.
4. Diluted virus (200 μl) of 1/2000 viral stock solution was added to each test compound (200 μl) and left to incubate at room temperature for the various times specified. (For the antiviral control, 40 μl of the diluted virus was added to 360 μl of citrate buffer.)
5. The reaction was terminated by the addition of cell infection media (3.6 ml), which diluted the reaction 10-fold.
6. The termination mixture was centrifuged (4000 rpm for 10 minutes) and the supernatant harvested.
7. The cell infection media in wells B4-B11 of the 96-well plate was removed. The supernatant (111 μl/well) was added to wells B8-B11, and the virus only control (1/2000 viral stock solution) was added to wells B4-B7. Both were plated in quadruplicate.
8. The plates were incubated at 37° C. and 5% CO2 for 2-3 days.
9. On day 2 or 3 post-infection, the plates were scored for vCPE and an HA assay was performed as per Retroscreen Virology Ltd. SOP VA018-02.

Controls utilized in the virucidal assay were:
Cell only control: cells not infected with virus. This is a negative control for vCPE and is also an indicator of cell quality.
Virus only control: cells infected with a 1/2000 dilution of the virus stock. This was a positive control for vCPE.
Diluent control: cells infected with virus that was pre-treated with sterile double-distilled water for the specified times. This was a negative control for the test compounds and assessed any antiviral effects of the diluent.
Antiviral control: cells infected with virus pre-treated with citrate buffer at pH3.5. This was a positive control for the test compounds.

For the virucidal assay only, the test compounds were prepared at double the concentrations than those described above. This is due to the 2-fold dilution they underwent when they were mixed with the virus.

R-100, R-101, and R-102 all exhibited time-dependent response toxicity against MDCK cells. R-100, R-101, and R-102 all exhibited a dose-response activity against Influenza A/Panama/2007/99. All the test concentrations of each test compound exhibited time-dependent response activity against Influenza A/Panama/2007/99. Only the highest test concentration (0.01% w/v) of each test compound exhibited significant reductions in virus titer at every incubation time tested.

The toxicity data generated shows that a time-response, and not a dose-response, was exhibited by the test compounds. This confirms earlier research that the incubation time rather than the test compound concentration is the determining factor of toxicity. It was also observed that the survivability of MDCK cells was also affected by the diluent control, as the values generated for the diluent control and the test compounds were similar.

After examining all the data examining toxicity, viral reduction, and therapeutic index, it was determined that there was a difference between the test compounds, but this difference was only marked when at a concentration of 0.01% w/v. As there was a difference between the toxicity of the test compounds, this suggested that the diluent, which remained consistent between the test compounds, has minimal toxicity. Toxicity and reductions in viral titer increased between R-100, R-101, and R-102 respectively. However small changes in percent toxicity for the 0.01% w/v concentration for all the test compounds had considerable impacts on the therapeutic index values.

In summary, R-102 at the highest concentration (0.01% w/v) affected the greatest reduction in viral titer with the highest therapeutic index.

### Virisorb Applications and Additional Examples

| | Example | Method of producing | Examples |
|---|---|---|---|
| 7 | Tissue & Towels | A gel comprised of water, the virucidal | The virucidal agent was a protonated |
| | | agent, and other ingredients known to the | montmorillonite added to deionized water |
| | | art is applied to the substrate that can be | in a concentration of 1% by weight. |
| | | composed of synthetic or natural fibers by | Between 0.0001% and 5% of the virucidal |
| | | either spraying, roll coating, dipping into a | agent, preferably 3% to 5%, is |
| | | trough containing the above described gel. | contemplated although higher |
| | | The final composition would contain the | percentages are useful. The slurry was |
| | | virucidal agent dispersed throughout. | uniformly sprayed onto a disposable |
| | | | “Bounty” towel in an amount equal to 5 |
| | | | times the weight of the original towel. The |
| | | | saturated towel was dried at 60° C. for 1 |
| | | | hour at which time it was determined that |
| | | | the water has been removed and the |
| | | | virucidal agent (protonated |
| | | | montmorillonite) remains on the towel. |
| | | | Other components that could be added to |
| | | | the gel include antimicrobials and |
| | | | disinfectants. |
| 8 | Masks and | The article of the above example is dried | The virucidal agent was a copper |
| | Disposable | by any number of methods well known to | exchanged montmorillonite added to |
| | Medical gowns. | the art. After drying the resultant fabric | deionized water in a concentration of 1% |
| | Air filters, | can be combined with another nonwoven | by weight. Between 0.0001% and 5%, |
| | | material using common laminating | preferably 3% to 5% of the virucidal agent |
| | | techniques. The outer layer of such a | is contemplated although higher |
| | | composition would contain the virucidal | percentages are useful. The slurry was |
| | | composition and can be further converted | uniformly sprayed onto a disposable 3M |
| | | into a disposal mask, air filter, medical | dust mask in an amount equal to 10 times |
| | | gown, bandage, bed pad, and various | the weight of the original mask. The |
| | | articles of clothing. | saturated mask was dried at 80° C. for 1 |
| | | | hour at which time it was determined that |
| | | | the water has been removed and the |
| | | | virucidal agent (copper montmorillonite) |
| | | | remained on the towel. Other |
| | | | components that could be added to the |
| | | | gel include antimicrobials, and |
| | | | disinfectants. |
| 9 | Wall paper | The article of the above composition is | |
| | | dried by any number of methods. The | |
| | | composition is combined with another | |
| | | fabric or paper through commonly known | |
| | | laminating methods. The second material | |
| | | containing, on one of its sides, an | |
| | | adhesive that can be activated by any | |
| | | number of solvents. Said composition can | |
| | | then be used in clean room environments | |
| | | as a virus resistant wall covering. | |
| 10 | Wet Wipes | A gel comprised of water, the virucidal | The virucidal agent was a silver |
| | | agent, and other ingredients useful for | exchanged montmorillonite added to |
| | | cleaning surfaces is applied to a substrate | deionized water in a concentration of 1% |
| | | composed of either synthetic or natural | by weight. Between 0.0001% and 5%, |
| | | fibers by either spraying, coating by roller | preferably 1% to 5% of the virucidal agent, |
| | | or slot die, dipping into a trough containing | is contemplated, although higher |
| | | the gel, gravure or flexographic printing, | percentages are useful. The slurry was |
| | | inkjet printing, and other means known to | 1% clay uniformly sprayed onto a |
| | | the art. Said composition is further | nonwoven substrate in an amount equal to |
| | | converted by cutting and folding into a wet | 20 times the weight of the original |
| | | wipe. The wet wipe can then be used to | nonwoven substrate. Other components |
| | | clean various surfaces depositing the gel | that could be added to the gel include |
| | | from the substrate to the surface, | antimicrobials, and disinfectants. |
| | | including human skin, animal skin, wood, | |
| | | metal, and plastic surfaces in hospitals, | |
| | | homes, and office buildings, schools, and | |
| | | similar institutions. Wet wipes could also | |
| | | be used to clean and sanitize medical | |
| | | instruments, such as surgical tools, bed | |
| | | pans, and trays. All surfaces treated with | |
| | | the wet wipe would have the virucidal | |
| | | properties of the virucidal agent. | |
| 11 | Paints for clean | A liquid composition comprised of water, | The virucidal agent in an amount of at |
| | rooms | the virucidal agent and other ingredients | least 0.01% by weight, e.g., 0.01 to 10%, |
| | | known to be useful in paint and coating | is added to a formula containing 10-40% |
| | | applications including but not limited to | pigments, 30-55% water, one or more |
| | | pigments, surfactants, emulsifiers, | latex compounds, such as, vinyl-acetate, |
| | | solvents such as binders composed of | vinyl-acrylate, acrylate, vinyl-acrylate- |
| | | vinyl acetate, vinyl acrylate, acrylate, | ethylene, and vinyl-ethylene, urethane- |
| | | urethane or combinations thereof; | acrylate emulsions in the amount ranging |
| | | epoxies, polyesters, and other setting | from 5-25%. The above composition |
| | | compounds as well as solvents useful for | can be applied to walls, floors, and other |
| | | enabling their compounding, are applied | surfaces. |
| | | to walls, floors, counter-tops with a roller, | |
| | | brush, or by air or airless spraying | |
| | | methods. The composition upon | |
| | | application will inactivate any viruses on | |
| | | the surfaces it ahs been applied to. | |
| | | Further after application, the composition | |
| | | will retain the ability to further inactivate | |
| | | any viruses that come in contact with the | |
| | | surfaces in the future. | |
| 12 | Laundry additives | The virucidal agent is combined with | |
| | | zeolites, surfactants, and other ingredients | |
| | | commonly used in a laundry detergent. | |
| | | The composition can then be used as a | |
| | | virucidal agent for cleaning washable | |
| | | materials. | |
| 13 | Absorbent mat | A plurality of fibers are combined with the | |
| | with antimicrobial | virucidal agent and alternatively absorbent | |
| | and virucidal | polymers, antimicrobials and anti- | |
| | capability | bacterials. Additional agents to reduce | |
| | | odor may also be included. The final mat | |
| | | is then capable of absorbing fluids and | |
| | | rendering them non infectious | |
| | | alternatively, the mat can be placed over | |
| | | spills of infectious materials and used to | |
| | | absorb these fluids and render them | |
| | | noninfectious. | |
| 14 | Carpet cleaners | The virucidal agent is combined with talc, | The sodium montmorillonite virucidal |
| | and upholstery | sodium bicarbonate, surfactants, | agent was combined in a weight amount |
| | | fragrances and other ingredients | of 70% with 15% talc and 15% sodium |
| | | commonly used in powdered carpet and | bicarbonate. The mixture was a light |
| | | upholstery cleaners. The composition can | colored free flowing powder and can be |
| | | then be used as a virucidal agent by | sprinkled on carpet or upholstery where it |
| | | pouring or sprinkling on the carpet and | will interact with any virus present, easily |
| | | upholstery where it will interact with the | removes the carpet cleaner and bound |
| | | virus and can be subsequently vacuumed | virus molecules as determined by removal |
| | | up. | of the light colored material. |
| 15 | Condom Coating | A gel comprised of water, the virucidal | To a coating solution comprised of |
| | | agent, anti-agent and other ingredients | glycerine, polyethylene glycol or a mixture |
| | | known to the art is applied to the condom | of water, a humectant and a thickener |
| | | prior to packaging. The final composition | such as hydroxylpropyl cellulose is added |
| | | would contain the virucidal agent | the virucidal agent in a concentration of at |
| | | dispersed throughout. In event of condom | least 0.001% up to 30 wt. %. The coating |
| | | failure, the virucidal agent would interact | solution is then placed on the condom to |
| | | with virus released by the male or virus | completely lubricate the surface. The |
| | | already present in the partner to prevent | mixture may also include anti-spermicidal |
| | | infection of either partner. | agents such as Nonoxynol-9. |
| 16 | Vaginal Gel | A gel, creme, or body heat dissolving | The virucidal agent is incorporated in a |
| | | tablet or suppository comprised of water, | water-based formulation that contains |
| | | the virucidal agent, and other ingredients | greater than 0.001% of the |
| | | known to the art is inserted into the vagina | Montmorillonite and includes thickeners |
| | | prior to sexual activity. The final | for the water, such as xanthane gum or |
| | | composition would contain the virucidal | Carbopol along with humectants like |
| | | agent dispersed throughout. The virucidal | glycerine and propylene glycol. |
| | | agent would interact with virus released by | Alternatively, the virucidal agent could be |
| | | the male or virus already present in the | dispersed in a non-aqueous vehicle like |
| | | partner to prevent infection of either | glycerine, propylene glycol or polyethylene |
| | | partner. The product could also be used | glycol. |
| | | in a douche format to cleanse vaginal area | |
| | | after sexual intercourse and deactivate | |
| | | viruses. | |
| 17 | Hand Sanitizer | A hand sanitizer gel comprised of water, | The formula contained from about 40% to |
| | | the virucidal agent, anti-microbial agent | about 70% by weight ethyl alcohol, 30-60% |
| | | and other ingredients known to the art is | water, glycerin, Carbomer and 1% by |
| | | applied to the hand to improve sanitation. | weight of the sodium montmorillonite |
| | | The final composition would contain the | virucidal agent. The virucidal agent can |
| | | virucidal agent dispersed throughout. | be in an amount of 0.001% to 15% by |
| | | Viricudal agent would inactivate virus | weight. The formula was rubbed on |
| | | present on the hands. | hands to provide for instant sanitization |
| | | | and inactivation of hand-held viruses. |
| 18 | Gastrointestinal | Virucidal agent our compounds are | A formulation can be a gastrointestinal |
| | Agent | ingested. In gastrointestinal tract, they | retention system in the form of tablet, |
| | | interact with viruses and prevent infection. | capsule or oral solution which may |
| | | When wastes are expelled, viruses are | additionally incorporate a mucoadhesive |
| | | retained on our materials and prevented | polymer. |
| | | from causing secondary infections. | |
| 19 | Nasal Lubricant | A solution/spray of the virucidal agent is | A gel comprised of water and the sodium |
| | | placed into nasal passages where it coats | Montmorillonite agent in a weight |
| | | nasal cells. When a virus contacts the | percentage from 0.00001% to 15%, more |
| | | virucidal agent, it is inactivated and | preferably 1-7%, is combined with non- |
| | | prevents infection | swelling sodium polyacrylate, know by the |
| | | | trade name CARBOPOL ®. Said gel is |
| | | | placed in a squeeze bottle with a nozzle in |
| | | | its top capable of being safely inserted |
| | | | into the nasal cavity. The gel is sprayed |
| | | | into the nasal passages by squeezing the |
| | | | bottle. The above gel may also contain |
| | | | one or more of the following materials — |
| | | | decongestants, such as Phenylephrine |
| | | | Hydrochloride, and other cold relief, |
| | | | menthol, camphor, sodium chloride, |
| | | | thimerosol and other ingredients known in |
| | | | the art. |
| 20 | Dialysis Filter | The virucidal agent is placed in a filter | The virucidal agent is present in the filter |
| | | canister and blood product is pumped | either as a 100% pure media or diluted |
| | | through the filter. The virucidal agent | 10-90% with a porosity aid. The blood |
| | | interacts with the virus present in the | product is circulated through the filter |
| | | blood product influent to reduce and | where the virucidal agent interacts with |
| | | maintain the viral count at an acceptable | the virus present in the blood product to |
| | | level. The acceptable level is dependent | maintain the viral count below an |
| | | on the virus desired to be removed. | acceptable level. It is envisioned that a |
| | | | protonated silver exchanged |
| | | | montmorillonite would be an effective |
| | | | virucidal product for dialysis filters. |
| 21 | Spill Containment | The virucidal agent is combined with other | The sodium montmorillonite virucidal |
| | | absorbent and adsorbent materials such | agent was combined in a weight |
| | | as vermiculite, sodium bentonite, oil | percentage of 50% with 30% oil adsorbent |
| | | adsorbents, polyacrylate superabsorbent | and 20% polyacrylate superabsorbent |
| | | polymers, and surfactants. In the event of | polymer. The mixture was a free flowing |
| | | a spill of a virus containing solution in a | powder and can be poured on an organic |
| | | medical associated laboratory, the | or aqueous based spill where it will |
| | | virucidal agent containing spill | interact with any virus present. |
| | | containment mixture is poured on the spill | Vacuuming easily removes the spill |
| | | area and the liquid as well as the virus is | containment agent as determined by |
| | | contained and cleaned up by shovel, or | removal of the brown colored material. |
| | | sweeping. | |

| | Example | Method of producing |
|---|---|---|
| 22 | Vaginal Inserts/STD's | A liquid composition comprised of water, the virucidal agent, and other |
| | | cosmetically and pharmaceutically acceptable ingredients such as |
| | | glycerin, sorbitol, ethyl alcohol, thickeners such as xanthan gum, and the |
| | | like, surfactants, such as lauryl sulfate, and the like. The composition can |
| | | then be used as a gel for applying on male genetalia, vaginal inserts and |
| | | nasal sprays. |
| 23 | Hand sanitizers | The composition of the above example can be combined with ethyl |
| | | alcohol, and/or other antimicrobials such as triclosan, and/or cetyl |
| | | pyridinium chloride and the like. This composition can be used as an |
| | | instant hand sanitizer with enhanced ability to inactivate viruses. |
| 24 | Nasal Gel/spray | The composition of example 22 can be inserted or sprayed into the nasal |
| | | passages |
| 25 | Cold Sore Treatment | The composition of example 22 can be applied to cold sores to aid in |
| | | reducing the duration of cold sores through inactivating the herpes virus. |
| 26 | Alternative Lip and genitalia | An anhydrous gel containing one or more of anhydrous ingredients |
| | protectant | including waxes, synthetic and natural oils, silicones, petrolatum and the |
| | | virucidal agent are mixed together. The compositing is melted and poured |
| | | into a mold, commonly used to form lip coating products. Upon cooling, |
| | | the materials are removed from the molds and can be used as lipsticks, lip |
| | | balms, vaginal inserts, and the like. |
| 27 | Emulsion | Water containing the virucidal agent, and surfactants and lipophilic |
| | | materials such as waxes, synthetic or natural oils, silicones, |
| | | hydrocarbons, and similar materials can be combined by mixing under |
| | | high shear to create an emulsion. This emulsion can be used directly on |
| | | human skin, animal skin and various surfaces as a virucidal agent. |
| | | Alternatively, the composition can be applied to substrates and dried to |
| | | create a filter, bandage and mask. In addition, the emulsion can be |
| | | applied to a substrate that is further converted into a wet wipe that can be |
| | | used to apply the virucidal composition to various surfaces. |
| 28 | Filter device for removing | The virucidal agent is placed in a cartridge that has a porous cover, or a |
| | virus from fluids | plurality of holes, that enables liquid to flow through the cartridge, but |
| | | retain the virucidal agent within it. The device can then be used to |
| | | inactivate viruses in the blood stream of animals or humans, water, and |
| | | any other liquid that may contain viruses. |
| 29 | Blood adsorbent with virus | The virucidal agent is combined with absorbent polymers and other anti |
| | inactivation | microbial or antibacterial agents, such as CPC, triclosan, and the like. |
| | | The powder is then capable of solidifying liquid and semi-solid wastes |
| | | from animals and humans and inactivating viruses present in the wastes, |
| | | eliminating the potential for spreading infectious diseases. |

### Example 30

### Antiviral Activity of Test Compounds against Feline Calcivirus

In this study, three different compositions of bentonite clay were studied (R-400, R-401, and R-402) to evaluate their antiviral efficacy against a Feline Calcivirus (a surrogate for Norovirus) (ATCC VR-782).

Test substances.

R-400: purified homolonic sodium bentonite mixture, purified in accordance with U.S. Pat. No. 6,050,509

R-401: purified homolonic hydrogen (protonated) bentonite mixture

R-402: purified homoionic hydrogen (protonated) bentonite #2 mixture

Each of the test substances were dispersed in double distilled water at a concentration of 0.1% (w/v) prior to use in the following assays.

Virus and Preparation of Stock Virus. The F-9 strain of the Feline Calcivirus stock virus was obtained from the American Type Culture collection, Manassas, Va. (ATCC VR-782). Stock virus was prepared by collecting the supernatant culture fluid from infected culture cells. The cells were disrupted and cell debris removed by centrifugation at 2000 RPM for five minutes at 4° C. The supernatant was removed, aliquoted, and the high titer stock virus was stored at ≦−70° C. until the day of use. On the day of use, five aliquots of stock virus (ATS Labs Lot FC-33) were removed, thawed, combined and refrigerated until use in the assay. The stock virus culture contained 5% fetal bovine serum (FBS) as the organic soil load. The stock virus tested demonstrated cytopathic effects (CPE) typical of Feline Calcivirus on feline kidney cells.

Test Cell Cultures. Cultures of feline kidney (CRFK) cells were originally obtained from the American Type Culture collection, Manassas, Va. (ATCC CCL-94). The cells were propagated, seeded into multiwell cell culture plates and maintained at 36-38° C. in a humidified atmosphere of 5-7% CO₂.

Test Medium. The test medium used in the following assays was Minimum Essential Medium (MEM), supplemented with 5% heat-inactivated fetal bovine serum (FBS), 10 μg/mL gentamicin, 100 U/mL penicillin, and 2.5 μg/mL amphotericin B.

Preparation of Test Substance. Each of R-400, R-401 and R-402 were shaken vigorously by hand for three minutes, aliquoted and utilized immediately in the following assays.

Treatment of Virus Suspension. For each exposure temperature (room temperature and 37° C.), a 4.5 mL aliquot of test substance was dispensed into separate sterile 15 mL conical tubes and mixed with a 0.5 mL aliquot of the stock virus suspension. The mixtures were vortex mixed for ten seconds and held for the remainder of the specified 30 second exposure time at room temperature (actual 24.5° C.) and at 37±1° C. (actual 37.0° C.). Immediately following the exposure time, a 0.1 mL aliquot was removed from each tube and the mixtures were titered by 10-fold serial dilution (0.1 mL+0.9 mL test medium). To decrease the test substance cytotoxicity, the first dilution was made in FBS with the remaining dilutions in test medium. Each assay was then assayed for the presence of the virus.

Treatment of Virus control: A 0.5 mL aliquot of stock virus suspension was exposed for 30 seconds to a 4.5 mL aliquot of test medium in lieu of test substance at exposure temperatures of 24.5° C. and 37.1° C. Immediately following the exposure time, a 0.1 mL aliquot was removed from each tube and the mixtures were titered by 10-fold serial dilution (0.1 mL+0.9 mL test medium). All controls employed the FBS neutralized as described in the Treatment of Virus Suspension section. The virus control titer was used as a baseline to compare the percent and log reductions of each test parameter following exposure to the test substances.

Cytotoxicity Controls. A 4.5 mL aliquot of each test substance was mixed with a 0.5 mL aliquot of test medium containing 5% FBS in lieu of virus and treated as previously described for each exposure temperature assayed. The cytotoxicity of the cell cultures was scored at the same time as virus-test substance and virus control cultures. Cytotoxicity was graded on the basis of cell viability as determined microscopically. Cellular alterations due to toxicity were graded and reported as toxic (T) if greater than or equal to 50% of the monolayer was affected.

Neutralization Controls. Each cytotoxicity control mixture was challenged with low titer stock virus to determine the dilution(s) of test substance at which virucidal activity, if any, was retained. Dilutions that showed virucidal activity were not considered in determining reduction of the virus by the test substance.

Neutralization Assay. As described above, 0.1 mL of each test and control parameter following the exposure time period was added to FBS (0.9 mL) followed immediately by 10-fold serial dilutions in test medium to stop the action of the test substance. To determine if the neutralizer chose for the assay was effective in diminishing the virucidal activity of the test substance, low titer stock virus was added to each dilution of the test substance-neutralizer mixture. The mixtures were assayed for the presence of virus.

Infectivity Assay. The CPFK cell line, which exhibits CPE in the presence of Feline Calcivirus, was used as the indicator cell line in the infectivity assays. Cells in multiwell culture dishes were inoculated in quadruplicate with 0.1 mL of the dilutions prepared from test and control groups. Uninfected indicator cell cultures (cell controls) were inoculated with test medium alone. The cultures were incubated at 31-35° C. in a humidified atmosphere of 5-7% CO₂ in sterile disposable cell culture lab ware. The cultures were microscopically scored periodically for seven days for the absence or presence of CPE, cytotoxicity, and for viability.

Results for Test Substances R-400, R-401 and R-402. Following a 30 second exposure time at room temperature (24.5° C.), test virus infectivity was detected in the virus-test substance mixture at 7.0 log₁₀. Test substance cytotoxicity was detected at 3.5 log₁₀. The neutralization control demonstrated that the test substance was neutralized at ≦2.5 log₁₀. Taking the cytotoxicity and neutralization control results into consideration, R-400 demonstrated an 82.2% reduction (at 24.5° C.) and a ≧96.8% reduction (at 37.0° C.) in viral titer following a 30 second exposure time to the virus. The log reductions in viral titer were 0.75 log₁₀ and ≧1.5 log₁₀, respectively. R-401 demonstrated a 43.8% reduction (at 24.5° C.) and a ≧96.8% reduction (at 37.0° C.) in viral titer following a 30 second exposure time to the virus. The log reduction in viral titers was 0.25 log₁₀ and ≧1.5 log₁₀, respectively. R-0402 demonstrated a 68.4% reduction (at 24.5° C.) and a ≧96.8% reduction (at 37.0° C.) in viral titer following a 30 second exposure time to the virus. The log reductions in viral titer were 0.5 log₁₀ and ≧1.5 log₁₀, respectively.

### Example 31

### Antiviral Activity of Test Compounds against Rotavirus

In this study, three different compositions of bentonite clay were studied (R-400, R-401, and R-402) to evaluate their adsorption and antiviral efficacy against Rotavirus.

Test substances.

R-400: purified homoionic sodium bentonite mixture, purified in accordance with U.S. Pat. No. 6,050,509

R-401: purified homoionic hydrogen (protonated) bentonite mixture

R-402: purified homoionic hydrogen (protonated) bentonite #2 mixture

Each of the test substances were dispersed in double distilled water at a concentration of 0.1% (w/v) prior to use in the following assays.

Virus and Preparation of Stock Virus. The WA strain of Rotavirus was obtained from the University of Ottawa, Ontario, Canada. Stock virus was prepared by collecting the supernatant culture fluid from infected culture cells. The cells were disrupted and cell debris removed by centrifugation at 2000 RPM for five minutes at approximately 4° C. The supernatant was removed, aliquoted, and the high titer stock virus was stored at ≦−70° C. until the day of use. On the day of use, five aliquots of stock virus (ATS Labs Lot XR-115) were removed, thawed, combined and refrigerated until use in the assay. The stock virus culture contained 5% fetal bovine serum (FBS) as the organic soil load. The stock virus tested demonstrated cytopathic effects (CPE) typical of Rotavirus on MA-104 cells.

Test Cell Cultures. Cultures of MA-104 (Rhesus monkey kidney) cells were originally obtained from Diagnostics Hybrids Inc., Athens, Ohio. The cells were propagated, seeded into multiwell cell culture plates and maintained at 36-38° C. in a humidified atmosphere of 5-7% CO₂.

Test Medium. The test medium used in the following assays was serum free Minimum Essential Medium (MEM), supplemented with 0.5 μg/mL trypsin, 2.0 mM L-glutamine, 10 μg/mL gentamicin, 100 U/mL penicillin, and 2.5 μg/mL amphotericin B.

Preparation of Test Substance. Each of R-400, R-401 and R-402 were shaken vigorously by hand for three minutes, aliquoted and utilized immediately in the following assays.

Treatment of Virus Suspension. For each exposure temperature (room temperature and 37° C.), a 4.5 mL aliquot of test substance was dispensed into separate sterile 15 mL conical tubes and mixed with a 0.5 mL aliquot of the stock virus suspension. The mixtures were vortex mixed for ten seconds and held for the remainder of the specified 30 second exposure time at room temperature (actual 24.5° C.) and at 37±1° C. (actual 38.0° C.). Immediately following the exposure time, a 0.01 mL aliquot was removed from each tube and the mixtures were titered by 10-fold serial dilution (0.1 mL+0.9 mL test medium). To decrease the test substance cytotoxicity, the first dilution was made in FBS with the remaining dilutions in test medium. Each assay was then assayed for the presence of the virus.

Treatment of Virus Control: A 0.5 mL aliquot of stock virus suspension was exposed for 30 seconds to a 4.5 mL aliquot of test medium in lieu of test substance at exposure temperatures of 24.5° C. and 38.0° C. Immediately following the exposure time, a 0.1 mL aliquot was removed from each tube and the mixtures were titered by 10-fold serial dilution (0.1 mL+0.9 mL test medium). All controls employed the FBS neutralized as described in the Treatment of Virus Suspension section. The virus control titer was used as a baseline to compare the percent and log reductions of each test parameter following exposure to the test substances.

Cytotoxicity Controls. A 4.5 mL aliquot of each test substance was mixed with a 0.5 mL aliquot of test medium containing 5% FBS in lieu of virus and treated as previously described for each exposure temperature assayed. The cytotoxicity of the cell cultures was scored at the same time as virus-test substance and virus control cultures. Cytotoxicity was graded on the basis of cell viability as determined microscopically. Cellular alterations due to toxicity were graded and reported as toxic (T) if greater than or equal to 50% of the monolayer was affected.

Neutralization Controls. Each cytotoxicity control mixture was challenged with low titer stock virus to determine the dilution(s) of test substance at which virucidal activity, if any, was retained. Dilutions that showed virucidal activity were not considered in determining reduction of the virus by the test substance.

Neutralization Assay. As described above, 0.1 mL of each test and control parameter following the exposure time period was added to FBS (0.9 mL) followed immediately by 10-fold serial dilutions in test medium to stop the action of the test substance. To determine if the neutralizer chose for the assay was effective in diminishing the virucidal activity of the test substance, low titer stock virus was added to each dilution of the test substance-neutralizer mixture. The mixtures were assayed for the presence of virus.

Infectivity Assay. The MA-104 cell line, which exhibits CPE in the presence of Rotavirus, was used as the indicator cell line in the infectivity assays. Cells in multiwell culture dishes were inoculated in quadruplicate with 0.1 mL of the dilutions prepared from test and control groups. Uninfected indicator cell cultures (cell controls) were inoculated with test medium alone. The inoculum was allowed to adsorb for 60 minutes at 36-38° C. in a humidified atmosphere of 5-7% CO₂. Following the adsorption period, a 1.0 mL aliquot of test medium was added to each well of the cell cultures, and the cultures were incubated at 36-38° C. in a humidified atmosphere of 5-7% CO₂ in sterile disposable cell culture lab ware. The cultures were microscopically scored periodically for seven days for the absence or presence of CPE, cytotoxicity, and for viability.

Infectivity Results for Test Substances R-400, R-401 and R-402. Following a 30 second exposure time at room temperature (24.5° C.), test virus infectivity was detected in the virus-test substance mixture at 7.25 log₁₀. Test substance cytotoxicity was detected at 3.5 log₁₀. The neutralization control demonstrated that the test substance was neutralized at ≦2.5 log₁₀. Taking the cytotoxicity and neutralization control results into consideration, R-400 demonstrated no reduction in viral titer following a 30 second exposure time to the virus at 24.5° C. or 38.0° C. R-401 also demonstrated no reduction in viral titer following a 30 second exposure time to the virus at 24.5° C. or 38.0° C. R-0402, however, demonstrated a 68.4% reduction (at 24.5° C.) and a ≧99.7% reduction (at 38.0° C.) in viral titer following a 30 second exposure time to the virus. The log reductions in viral titer were 0.5 log₁₀ and ≧2.5 log₁₀, respectively.

1. A method of inactivating a virus in the gastrointestinal tract of a mammalian subject comprising administering to said subject a composition comprising a layered phyllosilicate material in an amount effective for virus inactivation.

2. The method of claim 1, wherein the layered phyllosilicate material comprises at least 90% homoionic interlayer exchangeable cations, in relation to all interlayer exchangeable cations, and has a particle size less than 74 μm.

3. The method of claim 2, wherein the wherein the phyllosilicate material comprises interlayer exchangeable cations that are predominantly hydrogen cations.

4. The method of claim 2, wherein the layered phyllosilicate material comprises exfoliated platelets and/or tactoids of the layered phyllosilicate material.

5. The method of claim 1, wherein the composition further comprises a pharmaceutically acceptable carrier, diluent or adjuvant.

6. The method of claim 1, wherein the mammalian subject is human.

7. The method of claim 1, wherein the mammalian subject is an animal.

8. The method of claim 7, wherein the animal is selected from the group consisting of a horse, a cow, sheep, a pig, a llama, an alpaca, a goat, a dog, a cat, a mouse, a rat, a rabbit, a guinea pig and a hamster.

9. The method of claim 1, wherein the virus is an enterovirus.

10. The method of claim 9, wherein the virus is selected from the group consisting of polioviruses, coxsackieviruses, and echoviruses.

11. The method of claim of claim 1, wherein the virus is from a genus selected from the group consisting of calciviridae, norovirus and reoviridae.

12. The method of claim 11, wherein the virus is norovirus.

13. The method of claim 11, wherein the virus is feline calcivirus.

14. The method of claim 11, wherein the virus is rotavirus.

15. A method of treating a viral infection in the gastrointestinal tract of a mammalian subject comprising administering to said subject a composition comprising a layered phyllosilicate material and a pharmaceutically acceptable carrier.

16. The method of claim 15, wherein the layered phyllosilicate material comprises at least 90% homoionic interlayer exchangeable cations, in relation to all interlayer exchangeable cations, and has a particle size less than 74 μm.

17. The method of claims 16, wherein the phyllosilicate material comprises interlayer exchangeable cations that are predominantly hydrogen cations.

18. The method of claim 16, wherein the layered phyllosilicate material comprises exfoliated platelets and/or tactoids of the layered phyllosilicate material.

19. The method of claim 15, wherein the composition further comprises a pharmaceutically acceptable carrier, diluent or adjuvant.

20. The method of claim 15, wherein the mammalian subject is human.

21. The method of claim 15, wherein the mammalian subject is an animal.

22. The method of claim 21, wherein the animal is selected from the group consisting of a horse, a cow, sheep, a pig, a llama, an alpaca, a goat, a dog, a cat, a mouse, a rat, a rabbit, a guinea pig and a hamster.

23. The method of claim 15, wherein the viral infection is caused by an enterovirus.

24. The method of claim 23, wherein the viral infection is caused by a virus selected from the group consisting of polioviruses, coxsackieviruses, and echoviruses.

25. The method of claim of claim 15, wherein the viral infection is caused by a virus from a genus selected from the group consisting of calciviridae, norovirus and reoviridae.

26. The method of claim 25, wherein the virus is norovirus.

27. The method of claim 25, wherein the virus is feline calcivirus.

28. The method of claim 25, wherein the virus is rotavirus.

29. A method of delivering a therapeutic agent to a mammalian subject in need thereof comprising administering a composition comprising a therapeutic agent and a layered phyllosilicate material.

30. The method of claim 29, wherein said therapeutic agent is intercalated within the layered phyllosilicate material.

31. The method of claim 29, wherein the therapeutic agent is selected from the group consisting of a nucleic acid, a protein, and a small molecule drug.

32. The method of claim 29, wherein the therapeutic agent is selected from the group consisting of colloidal silver, an antisense nucleotide, a thrombin inhibitor, an antithrombogenic agent, a tissue plasminogen activator, a thrombolytic agent, a fibrinolytic agent, a vasospasm inhibitor, a calcium channel blocker, a nitrate, a nitric oxide promoter, a vasodilator, an antimicrobial agent, an antibiotic, an antiplatelet agent, an antimitotic, a microtubule inhibitor, an actin inhibitor, a remodeling inhibitor, an agent for molecular genetic intervention, a cell cycle inhibitor, an inhibitor of the surface glycoprotein receptor, an antimetabolite, an antiproliferative agent, an anti-cancer chemotherapeutic agent, an anti-inflammatory steroid, an immunosuppressive agent, an antibiotic, a radiotherapeutic agent, iodine-containing compounds, barium-containing compounds, a heavy metal functioning as a radiopaque agent, a peptide, a protein, an enzyme, an extracellular matrix component, a cellular component, a biologic agent, an angiotensin converting enzyme (ACE) inhibitor, ascorbic acid, a free radical scavenger, an iron chelator, and an antioxidant.

33. A patch comprising a pad material having an upper surface and lower surface, an adhesive on the lower surface, and a therapeutic composition, wherein the therapeutic composition comprises a layered phyllosilicate material.

34. A surgical suturing thread coated or impregnated with a composition, wherein said composition comprises a layered phyllosilicate material.

35. A method of promoting the absorption of a therapeutic agent through the mucosal membranes in a mammalian subject, comprising administering to said subject a composition comprising a therapeutic agent, a layered phyllosilicate material and pharmaceutically acceptable carrier, diluent or excipient.

36. A method of delivering a diagnostic agent to a biological fluid or a subject comprising administering a composition comprising a diagnostic agent and a layered phyllosilicate material.

37. A method of inactivating a virus in waste expelled from a mammal comprising administering to said mammal a composition comprising a layered phyllosilicate material and pharmaceutically acceptable carrier, diluent or excipient in an amount effective for virus inactivation.

38. The method of claim 37, wherein said waste is fecal matter.

39. The method of claim 37, wherein said waste is urine.

40. A method of preventing viral resistance to an anti-viral material, comprising contacting a virus with a material that interacts with the surface molecules of said virus, wherein said interaction is a mechanism selected from the group consisting of adsorption, ionic complexing, electrostatic complexing, chelation, hydrogen bonding, ion-dipole, dipole/dipole, Van Der Waals forces and combinations thereof.

41. The method of claim 40, wherein said material is a layered phyllosilicate material.

42. A method of preventing viral resistance to an anti-viral material comprising contacting a virus with a composition comprising a layered phyllosilicate material in an amount effective to prevent viral resistance.
